SEQUENCE LISTING (64 sequences)

SEQ ID NO: 1 - protein, 223 aa, artificial
  Rat anionic trypsin II
IVGGYTCQENSVPYQVSLNSGYHFCGGSLINDQWVVSAAHCYKSRIQVRLGEHNINVLEGNEQFVNAAKIIKHPNFDRKTLNNDIMLIKLSSPVKLNARVATVALPSSCAPAGTQCLISGWGNTLSSGVNEPDLLQCLDAPLLPQADCEASYPGKITDNMVCVGFLEGGKDSCQGDSGGPVVCNGELQGIVSWGYGCALPDNPGVYTKVCNYVDWIQDTIAAN

SEQ ID NO: 2 - protein, 245 aa, artificial
  anti-Her2-Fab-LC_RRKH-HC_YRAH, Heavy chain
EVKLQESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCADKPGGYRAHAGEQKLISEEDL

SEQ ID NO: 3 - protein, 225 aa, artificial
  anti-Her2-Fab- LC_RRKH-HC_YRAH, Light chain
DIELTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLSPGGRRKHAG

SEQ ID NO: 4 - protein, 9 aa, artificial
  Polypeptide
PGGYRAHAG

SEQ ID NO: 5 - protein, 9 aa, artificial
  Polypeptide
PGGFRAHAG

SEQ ID NO: 6 - protein, 9 aa, artificial
  Polypeptide
PGGWRAHAG

SEQ ID NO: 7 - protein, 9 aa, artificial
  Polypeptide
PGGLRAHAG

SEQ ID NO: 8 - protein, 9 aa, artificial
  Polypeptide
PGGDRAHAG

SEQ ID NO: 9 - protein, 9 aa, artificial
  Polypeptide
PGGRRAHAG

SEQ ID NO: 10 - protein, 9 aa, artificial
  Polypeptide
PGGARAHAG

SEQ ID NO: 11 - protein, 9 aa, artificial
  Polypeptide
PGGYAAHAG

SEQ ID NO: 12 - protein, 9 aa, artificial
  Polypeptide
PGGYDAHAG

SEQ ID NO: 13 - protein, 9 aa, artificial
  Polypeptide
PGGYEAHAG

SEQ ID NO: 14 - protein, 9 aa, artificial
  Polypeptide
PGGYKAHAG

SEQ ID NO: 15 - protein, 9 aa, artificial
  Polypeptide
PGGYRAAAG

SEQ ID NO: 16 - protein, 9 aa, artificial
  Polypeptide
PGGYRANAG

SEQ ID NO: 17 - protein, 9 aa, artificial
  Polypeptide
PGGYRAKAG

SEQ ID NO: 18 - protein, 9 aa, artificial
  Polypeptide
PGGYRADAG

SEQ ID NO: 19 - protein, 9 aa, artificial
  Polypeptide
PGGYRKHAG

SEQ ID NO: 20 - protein, 9 aa, artificial
  Polypeptide
PGGFRKHAG

SEQ ID NO: 21 - protein, 9 aa, artificial
  Polypeptide
PGGWRKHAG

SEQ ID NO: 22 - protein, 9 aa, artificial
  Polypeptide
PGGLRKHAG

SEQ ID NO: 23 - protein, 9 aa, artificial
  Polypeptide
PGGDRKHAG

SEQ ID NO: 24 - protein, 9 aa, artificial
  Polypeptide
PGGRRKHAG

SEQ ID NO: 25 - protein, 9 aa, artificial
  Polypeptide
PGGARKHAG

SEQ ID NO: 26 - protein, 9 aa, artificial
  Polypeptide
PGGYAKHAG

SEQ ID NO: 27 - protein, 9 aa, artificial
  Polypeptide
PGGYDKHAG

SEQ ID NO: 28 - protein, 9 aa, artificial
  Polypeptide
PGGYMKHAG

SEQ ID NO: 29 - protein, 9 aa, artificial
  Polypeptide
PGGYRRHAG

SEQ ID NO: 30 - protein, 9 aa, artificial
  Polypeptide
PGGYRKAAG

SEQ ID NO: 31 - protein, 9 aa, artificial
  Polypeptide
PGGYRKNAG

SEQ ID NO: 32 - protein, 9 aa, artificial
  Polypeptide
PGGYRKKAG

SEQ ID NO: 33 - protein, 9 aa, artificial
  Polypeptide
PGGYRKDAG

SEQ ID NO: 34 - protein, 9 aa, artificial
  Polypeptide
PGGYKRKAG

SEQ ID NO: 35 - protein, 9 aa, artificial
  Polypeptide
PGGRMKHAG

SEQ ID NO: 36 - protein, 5 aa, artificial
  Polypeptide
RAHAK

SEQ ID NO: 37 - protein, 5 aa, artificial
  Polypeptide
AAHAK

SEQ ID NO: 38 - protein, 5 aa, artificial
  Polypeptide
DAHAK

SEQ ID NO: 39 - protein, 5 aa, artificial
  Polypeptide
EAHAK

SEQ ID NO: 40 - protein, 5 aa, artificial
  Polypeptide
KAHAK

SEQ ID NO: 41 - protein, 5 aa, artificial
  Polypeptide
RAAAK

SEQ ID NO: 42 - protein, 5 aa, artificial
  Polypeptide
RANAK

SEQ ID NO: 43 - protein, 5 aa, artificial
  Polypeptide
RAKAK

SEQ ID NO: 44 - protein, 5 aa, artificial
  Polypeptide
RADAK

SEQ ID NO: 45 - protein, 5 aa, artificial
  Polypeptide
RKHAK

SEQ ID NO: 46 - protein, 5 aa, artificial
  Polypeptide
AKHAK

SEQ ID NO: 47 - protein, 5 aa, artificial
  Polypeptide
DKHAK

SEQ ID NO: 48 - protein, 5 aa, artificial
  Polypeptide
MKHAK

SEQ ID NO: 49 - protein, 5 aa, artificial
  Polypeptide
RRHAK

SEQ ID NO: 50 - protein, 5 aa, artificial
  Polypeptide
RKAAK

SEQ ID NO: 51 - protein, 5 aa, artificial
  Polypeptide
RKNAK

SEQ ID NO: 52 - protein, 5 aa, artificial
  Polypeptide
RKKAK

SEQ ID NO: 53 - protein, 5 aa, artificial
  Polypeptide
RKDAK

SEQ ID NO: 54 - protein, 5 aa, artificial
  Polypeptide
KRKAK

SEQ ID NO: 55 - protein, 5 aa, artificial
  Polypeptide
RAHAK

SEQ ID NO: 56 - protein, 5 aa, artificial
  Polypeptide
RKHAK

SEQ ID NO: 57 - protein, 5 aa, artificial
  Polypeptide
RKKAK

SEQ ID NO: 58 - protein, 5 aa, artificial
  Polypeptide
RKAAK

SEQ ID NO: 59 - protein, 5 aa, artificial
  Polypeptide
KRKAK

SEQ ID NO: 60 - protein, 5 aa, artificial
  Polypeptide
RKHAK

SEQ ID NO: 61 - protein, 8 aa, artificial
  Polypeptide
RKKAKAAK

SEQ ID NO: 62 - protein, 5 aa, artificial
  Polypeptide
RKKAK

SEQ ID NO: 63 - protein, 5 aa, artificial
  Polypeptide
RKKAK

SEQ ID NO: 64 - DNA, 1552 bp, artificial
  Gene sequence of anti-Her2-Fab-LC_RRKH-HC_YRAH
atgaagaaaaccgcgattgcgattgcggtggcgctggcgggctttgcgaccgtggcgcaggcggatattgaactgacccagagcccgagcagcctgagcgcgagcgtgggcgatcgcgtgaccattacctgccgcgcgagccaggatgtgaacaccgcggtggcgtggtatcagcagaaaccgggcaaagcgccgaaactgctgatttatagcgcgagctttctgtatagcggcgtgccgagccgctttagcggcagccgcagcggcaccgattttaccctgaccattagcagcctgcagccggaagattttgcgacctattattgccagcagcattataccaccccgccgacctttggccagggcaccaaactggaaattaaacgcaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaatcttttaaccgcggcgaatgcctgagccccggaggacgccgcaaacatgcgggctgaggaggaaaaaaaaatgaaaaagacagctatcgcaattgcagtggcgctagctggtttcgccaccgtggcgcaagctgaagtgaaactgcaggaaagcggtggtggtctggtgcagccgggtggtagcctgcgcctgagctgcgcggcgagcggctttaacattaaagatacctatattcattgggtgcgccaggcgccgggcaaaggcctggaatgggtggcgcgcatttatccgaccaacggctatacccgctatgcggatagcgtgaaaggccgctttaccattagcgcggataccagcaaaaacaccgcgtatctgcagatgaacagcctgcgcgcggaagataccgcggtgtattattgcagccgctggggcggcgatggcttttatgcgatggattattggggccagggcaccctggtgaccgtgagcagcgcgagcaccaaaggcccgagcgtgtttccgctggcgccgagcagcaaaagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatatttgcaacgtgaaccataaaccgagcaacaccaaagtggataaaaaagtggaaccgaaaagctgcgcggataaacccggaggatatcgcgcgcatgcgggcgaacagaaactgattagcgaagaagatctg

(19) United States
(12) Patent Application Publication
(10) Pub. No.: US 2022/0235344 A1
(43) Pub. Date: Jul. 28, 2022

(54) TRYPSIN VARIANTS WITH IMPROVED ENZYMATIC PROPERTIES

(71) Applicant: BIOPHARMA TRANSLATIONSINSTITUT DESSAU FORSCHUNGS GMBH, Dessau-Rosslau (DE)

(72) Inventors: René Wartner, Leipzig (DE); Marcus Boehme, Halle (DE); Frank Bordusa, Braunsbedra (DE); Andreas Hans Simon, Halle (DE); Thomas Richter, Dessau-Rosslau (DE)

(21) Appl. No.: 17/416,975

(22) PCT Filed: Dec. 19, 2019
(86) PCT No.: PCT/EP2019/086425

§ 371 Date: Jun. 21, 2021

(30) Foreign Application Priority Data
Dec. 19, 2018 (EP) 18214031.9

(51) Int. Cl. C12N 9/76

(52) U.S. Cl. CPC C12N 9/6427; C12Y 304/21004

(57) ABSTRACT

The present invention relates to trypsin variants with improved enzymatic properties, and particularly relates to a mutated trypsin comprising an amino acid substitution at least at two amino acid positions leading to an increased affinity for the nucleophilic substrate and/or at least at two amino acid positions leading to a reduced hydrolysis activity.

### FIELD OF THE INVENTION

The present invention relates to trypsin variants with improved enzymatic properties.

### BACKGROUND OF THE INVENTION

There is a high demand to provide polypeptides with covalent modifications and methods to introduce specific covalent modifications to polypeptides.

In addition to the various purely chemical methods, which are generally non-regiospecific or lead to global modifications of the polypeptide, there are also molecular biological and enzymatic methods, or combinations of chemical and enzymatic methods for the site-specific modification of polypeptides.

A site-specific modification of polypeptides can only occur in exceptional cases without prior manipulation of the corresponding polypeptide at the genetic level. For example, recognition sequences for subsequent enzyme-catalyzed modification are integrated at the genetic level into the polypeptide sequence, which can be used for position-specific modification (the position of the introduced label is determined by the position of the recognition sequence).

Beside the single modification of polypeptides in a site-specific manner, an orthogonal dual-modification of these by utilizing enzymes of only one origin represents a novelty which cannot be achieved applying current technologies. In the context of orthogonal dual-modification, the term “orthogonal” refers to the modification of a polypeptide upon two varying recognition sequences using two different biocatalysts of the same origin without significant cross reactivity.

Enzymatic methods for modifying polypeptides use intrinsic properties of enzymes such as the recognition of certain amino acid sequences or functionalities after the introduction of the corresponding recognition sequences by means of site-directed mutagenesis. Regiospecificity is generated by the high substrate specificity of the respective enzymes. The fact that each polypeptide modified in this way has only one recognition sequence gives the method a regio- and chemoselective character, since usually only one amino acid is modified within a consensus sequence.

Proteases can be useful enzymes for the modification of polypeptides. Trypsin is a serine protease which specifically cleaves carboxy-terminal of basic amino acid residues. The active site consists of Ser195, Asp102 and His57 (catalytic triad). Ser195 forms an acyl enzyme intermediate with the substrate to be cleaved and is thus significantly involved in the protease reactivity. This acyl enzyme intermediate can be attacked by variable nucleophiles such as water (peptide hydrolysis), amines (peptide aminolysis), alcohols and thiols (peptide (thio)esterification). By forming a covalent acyl enzyme intermediate, the serine protease trypsin thus meets all the requirements for kinetically controlled acyl transfer.

In contrast to peptide cleavage, peptide bond linkage is a two-substrate reaction. The acyl donor binds at the S-binding site of the enzyme, while the acyl acceptor interacts with the S′ binding region.

The C-terminal modification of polypeptides via stable amide bonds is based on transamidation. The C-terminal end region of the polypeptide to be labeled forms the acyl enzyme intermediate with the trypsin variant, which can then be attacked nucleophilically by the labeled acyl acceptor.

In WO 2006/015879 A1 a trypsin variant K60E/D189K/N143H/E151H (Trypsiligase I) is described which recognizes histidine side chains zinc-ion induced in the P₂′-position of a peptide with the restriction site -Tyr-Arg-His- hydrolyses specifically the recognition sequence -YRH- between amino acids tyrosine und arginine in the presence of zinc-ions.

In EP 18 205 212 trypsin variants comprising an amino acid substitution both at position K60 and D189, and at least one more amino acid substitution at position Y39 or Y59 are described. A preferred trypsin variant described is Y39H/Y59H/K60E/D189K (Trypsiligase II). EP 18 205 212 further relates to the use of a polypeptide comprising a target polypeptide and a restriction site peptide comprising the recognition site Tyr-Arg-Xaa-His, wherein Xaa is any amino acid, wherein said restriction site peptide overlaps with the target polypeptide by the amino acid Tyr at the C-terminal end of said target polypeptide as a substrate of a mutated trypsin as described in EP 18 205 212. Further a method for preparing a C-terminal transamidated target polypeptide and a method for preparing an N-terminal transacylated target polypeptide is provided.

In view of these trypsin variants there is a need for providing variants with improved synthetic properties favoring the peptide aminolysis over hydrolysis within the deacylation step of the transamidation reaction as well as an independence of metal ions.

### SUMMARY OF THE INVENTION

The inventors of the present invention have found trypsin variants comprising an amino acid substitution at least at two amino acid positions leading to an increased affinity for the nucleophilic substrate and/or at least at two amino acid positions leading to a reduced hydrolysis activity.

In a preferred embodiment the trypsin variants comprise either an amino acid substitution at least at two amino acid positions selected from group 1 comprising H40, A55, S214, G219, A221, preferably further comprising an amino acid substitution at least at one amino acid position of group 2 comprising R96, K97, L99, N143, E151, S190, Q192; or an amino acid substitution at least at one amino acid position selected from group 1 comprising H40, A55, S214, G219, A221 and an amino acid substitution at least at one amino acid position of group 2 comprising R96, K97, L99, N143, E151, S190, Q192; or
an amino acid substitution at least at one amino acid position selected from group 1 comprising H40, A55, S214, G219, A221 and an amino acid substitution at least at two amino acid position of group 2 comprising R96, K97, L99, N143, E151, S190, Q192; or
an amino acid substitution at least at two amino acid position selected from group 1 comprising H40, A55, S214, G219, A221 and an amino acid substitution at least at two amino acid position of group 2 comprising R96, K97, L99, N143, E151, S190, Q192; or
an amino acid substitution at least at three amino acid position selected from group 1 comprising H40, A55, S214, G219, A221 and an amino acid substitution at least at one amino acid position of group 2 comprising R96, K97, L99, N143, E151, S190, Q192; or
an amino acid substitution at least at three amino acid position selected from group 1 comprising H40, A55, S214, G219, A221 and an amino acid substitution at least at two amino acid position of group 2 comprising R96, K97, L99, N143, E151, S190, Q192; or
an amino acid substitution at least at three amino acid position selected from group 1 comprising H40, A55, S214, G219, A221 and an amino acid substitution at least at three amino acid position of group 2 comprising R96, K97, L99, N143, E151, S190, Q192.

The present invention further refers to the use of two different trypsin enzymes for an orthogonal dual-modification upon two different recognition sequences and to a method for orthogonal dual-modification of a substrate using two different trypsin variants.

In a preferred embodiment the use of two different trypsin enzymes for an orthogonal dual-modification upon two different recognition sequences uses as the first enzyme trypsin variant A2C8 and as the second enzyme trypsin variant K7F11, or the first enzyme is trypsin variant A2C8 and the second enzyme is trypsin variant K7F11_H39Y/H59Y/K189D, or the first enzyme is Trypsiligase II and the second enzyme is trypsin variant A2C8, or the first enzyme is Trypsiligase II and the second enzyme is trypsin variant K7F11, or the first enzyme is trypsin variant K7F11_H39Y/H59Y/K189D and the second enzyme is trypsin variant A2C8, or the first enzyme is trypsin variant K7F11_H39Y/H59Y/K189D and the second enzyme is trypsin variant K7F11.

In a preferred embodiment the method for orthogonal dual-modification of a substrate comprises the following steps: a) providing a substrate for orthogonal dual-modification, b) modifying the substrate using a first trypsin variant recognizing a first recognition sequence, c) modifying the substrate using a second trypsin variant recognizing a second recognition sequence. Preferably the first or second trypsin variant is selected from the group comprising Trypsiligase II, trypsin variant A2C8, trypsin variant K7F11, trypsin variant K7F11_H39Y/H59Y/K189D.

### DETAILED DESCRIPTION

To facilitate an understanding of the invention, a brief discussion of the terminology used in connection with the invention will be provided. The present disclosure uses the terminology of Schechter, J., and Berger, A., Biochem. Biophys. Res. Commun. 27 (1967) 157-162, to describe the location of various amino acid residues on the peptide substrate and individual binding sites within the active site of a corresponding proteolytic enzyme.

According to the terminology proposed by Schechter, J. and Berger, A., supra, the amino acid residues of the peptide substrate are designated by the letter “P”. The amino acids of the substrate on the N-terminal side of the peptide bond to be cleaved (the “cleavage site” or “recognition site”) are designated Pₙ . . . P₃, P₂, P₁ with Pₙ being the amino acid residue furthest from the cleavage site. Amino acid residues of the peptide substrate on the C-terminal side of cleavage site are designated P₁′, P₂′, P₃′, . . . Pₙ′ with Pₙ′ being the amino acid residue furthest from the cleavage site. Hence, the bond which is to be cleaved (the “cleavage site” or “recognition site”) is the P₁-P₁′ bond.

The generic formula for the amino acids of the substrate of an endopeptidase (like for example trypsin) is as follows:

Pₙ-P₃-P₂-P₁-P₁′-P₂′-P₃′Pₙ′

The designation of the substrate binding sites of an endopeptidase is analogous to the designation of amino acid residues of the peptide substrate. However, the binding sub-sites of an endopeptidase are designated by the letter “S” and can include more than one amino acid residue. The substrate binding sites for the amino acids on the N-terminal site of the cleavage site are labeled Sₙ . . . , S₃, S₂, S₁. The substrate binding sub site for the amino acids on the carboxy side of the cleavage site are designated S₁′, S₂′, S₃′, . . . Sₙ′. Hence, in an endopeptidase, the S₁′ sub site interacts with the P₁′ group of the peptide substrate and the incoming nucleophile.

A generic formula for describing substrate binding sites of an endopeptidase is:

Sₙ-S₃-S₂-S₁-S₁′-S₂′-S₃′-Sₙ′

The S₁ binding site binds the side chain of the penultimate amino acid, P₁, of the peptide substrate, in case of a trypsin variant according to this invention the amino acid Tyr. The S₁′ binding site interacts with the side chain of P₁′, in the present case with Arg. Likewise, the S₂′ binding site interacts with the side chain of the Xaa residue in position P₂′.

The term “variant” refers to polypeptides having amino acid sequences that differ to some extent from a native polypeptide sequence. Ordinarily, a variant amino acid sequence will possess at least about 80% homology with the corresponding parent trypsin sequence, and preferably, it will be at least about 90%, more preferably at least about 95% homologous with such corresponding parent trypsin sequence. The amino acid sequence variants possess substitutions, deletions, and/or insertions at certain positions within the amino acid sequence of the native amino acid sequence. Preferably sequence homology will be at least 96% or 97%.

“Homology” is defined as the percentage of residues within the amino acid sequence variant that are identical after aligning the sequences and introducing gaps, if necessary, to achieve the maximum percent homology. Methods and computer programs for the alignment are well known in the art. One such computer program is “Align 2,” authored by Genentech, Inc., which was filed with user documentation in the United States Copyright Office, Washington, D.C. 20559, on Dec. 10, 1991.

Mutated Trypsin

A first aspect of the present invention provides a mutated trypsin comprising an amino acid substitution at least at one amino acid position selected from the group comprising H40, A55, R96, K97, L99, N143, E151, S190, Q192, S214, G219, A221 according to the chymotrypsin nomenclature which corresponds to positions 23, 38, 78, 79, 81, 123, 131, 172, 174, 192, 196 and 198 respectively, of the trypsin sequence given in SEQ ID NO:1.

The skilled artisan is familiar with the so-called chymotrypsin nomenclature as, e.g., described in Hartley, B. S., and Shotton, D. M., The Enzymes, P. D. Boyer (ed.), Vol. 3, (1971), pp. 323-373 and will have no problem in aligning the positions of a variant trypsin with positions given according to the chymotrypsin nomenclature to the corresponding ones of the trypsin sequence of SEQ ID No: 1.

In a preferred embodiment the mutated trypsin comprises additional amino acid substitutions at both position K60 and D189, and at least one more amino acid substitution at position Y39 or Y59.

Position 39 according to chymotrypsin nomenclature corresponds to position 22 of the sequence of mature anionic rat trypsin II from Rattus norvegicus as given in SEQ ID NO: 1.

Position 59 according to chymotrypsin nomenclature corresponds to position 42 of the sequence of mature anionic rat trypsin II from Rattus norvegicus as given in SEQ ID NO: 1.

Position 60 according to chymotrypsin nomenclature corresponds to position 43 of the sequence of mature anionic rat trypsin II from Rattus norvegicus as given in SEQ ID NO: 1.

Position 189 according to chymotrypsin nomenclature corresponds to position 171 of the sequence of mature anionic rat trypsin II from Rattus norvegicus as given in SEQ ID NO: 1.

Since the skilled artisan is used to express positions referring to the chymotrypsin nomenclature, therefore, in the following the references to specific sequence position, e.g., position K60 or simply position 60 are exclusively based on positions according to the chymotrypsin nomenclature.

In a further preferred embodiment, the mutated trypsin comprises additional amino acid substitutions at both position K60 and D189, and at least one more amino acid substitution by histidine at position N143 or position E151 according to the chymotrypsin nomenclature which corresponds to positions 43, 171, 123, and 131, respectively, of the sequence given in SEQ ID NO:1.

Position 60 according to chymotrypsin nomenclature corresponds to position 43 of the sequence of mature anionic rat trypsin II from Rattus norvegicus as given in SEQ ID NO: 1.

Position 143 according to chymotrypsin nomenclature corresponds to position 123 of the sequence of mature anionic rat trypsin II from Rattus norvegicus as given in SEQ ID NO: 1.

Position 151 according to chymotrypsin nomenclature corresponds to position 131 of the sequence of mature anionic rat trypsin II from Rattus norvegicus as given in SEQ ID NO: 1.

Position 189 according to chymotrypsin nomenclature corresponds to position 171 of the sequence of mature anionic rat trypsin II from Rattus norvegicus as given in SEQ ID NO: 1.

To identify relevant mutation sites in trypsin enzymes and to provide variants with improved properties the inventors started with two independent enzyme libraries based on trypsin variant K60E/N143H/E151H/D189K (Trypsiligase I).

For the generation of library A the amino acid positions H40, A55, K97, L99, S190 and Q192 were randomized, while for library B the positions D95, R96, L99, S214, G219 and A221 were randomized. Selection by phage display with subsequent ELISA-based screening resulted in Trypsiligase I variant 2G10 for library A and variant 1C11 for library B as the best variants in terms of synthetic potential.

Enzyme kinetic analyses of these two variants (see Table 1 and FIG. 1) have shown that the optimization of the synthetic potential has different causes. Variant 2G10 (Trypsiligase I+H40P, A55S, K97D, L99F, S190S, Q192E) shows a strongly increased affinity for the nucleophilic RH substrate compared to the initial enzyme Trypsiligase I, whereby it is preferentially integrated as a nucleophile in competition to water and thus leads to aminolysis (=desired product formation) instead of undesired hydrolysis.

The opposite is the variant 1C11 (Trypsiligase I+R96V, L99F, S214G, G219S, A221G), which shows a hydrolysis activity reduced by a factor of 20 compared to Trypsiligase I, whereby the relationship between aminolysis and hydrolysis is strongly shifted to the side of aminolysis. An improved affinity for the nucleophile could not be shown for this variant.

### BRIEF DESCRIPTION OF THE FIGURES

FIG. 1: Time course of product formation for a transamidation reaction catalyzed by Trypsiligase I as well as the improved variants 2G10, 1C11 and the hybrid variant. Reaction conditions: 15 μM Bz-AAYRHAAG-OH (acyl donor), 30 μM H-RHAK-OH (acyl acceptor), 0.5-2.1 μM trypsin variant, 100 mM HEPES/NaOH pH 7.8, 0.1 mM ZnCl2, 100 mM NaCl, 10 mM CaCl2, T=30° C. UPLC analysis: Waters Acquity Ultra Performance LC, C18-column, gradient 5-40% acetonitrile, 4 min, detection at 254 nm.

FIG. 2: Substrate specificity data of chosen variants identified during phage-display selection and screening out of Trypsiligase II-library. 100 μM acyl donor (Bz-PGGXaaXaaXaaXaaAG-OH); 200 μM acyl acceptor (H-XaaXaaXaaAK(DNP)-OH); 1-5 μM enzyme variant; 100 mM HEPES pH7.8, 100 mM NaCl, 10 mM CaCl₂, 0.1 mM ZnCl₂, T=30° C. UPLC analysis: Waters Acquity Ultra Performance LC, C18-column, gradient 5-60% acetonitrile, 5 min, detection at 360 nm; (k_(cat,AL)\hat{ }app): apparent turnover rate for the aminolysis reaction. Amino acid variation of acyl donor/acyl acceptor pairs at Xaa-positions are indicated at the y-axis, e.g. YRAH relates to the acyl donor Bz-PGGYRAHAG-OH with the corresponding acyl acceptor H-RAHAK(DNP)-OH.

FIG. 3: Time course of product formation for a transamidation reaction catalyzed by variant A2C8, A2C8_H39Y, A2C8_H59Y and A2C8_H39Y/H59Y. Reaction conditions: 100 μM Bz PGGYRKKAG-OH (acyl donor), 200 μM H-RKKAK-OH (acyl acceptor), 1 μM trypsin variant, 100 mM HEPES/NaOH pH 7.8, 100 mM NaCl, 10 mM CaCl₂, T=30° C. UPLC analysis: Waters Acquity Ultra Performance LC, C18-column, gradient 5-40% acetonitrile, 4 min, detection at 254 nm.

FIG. 4: Substrate specificity data of chosen variants identified during phage-display selection and screening out of Trypsiligase II-library. 100 μM acyl donor (Bz-PGGXaaXaaXaaXaaAG-OH); 200 μM acyl acceptor (H-XaaXaaXaaAK(DNP)-OH); 1-5 μM enzyme variant; 100 mM HEPES pH7.8, 100 mM NaCl, 10 mM CaCl₂, 0.1 mM ZnCl₂, T=30° C.; UPLC analysis: Waters Acquity Ultra Performance LC, C18-column, gradient 5-60% acetonitrile, 5 min, detection at 360 nm; (k_(cat,AL)\hat{ }app): apparent turnover rate for the aminolysis reaction. Amino acid variation of acyl donor/acyl acceptor pairs at Xaa-positions are indicated at the y-axis, e.g. YRAH relates to the acyl donor Bz-PGGYRAHAG-OH with the corresponding acyl acceptor H-RAHAK(DNP)-OH.

FIG. 5: Time course of the product formation for a transamidation reaction catalyzed by the variant A2C8_H39Y/H59Y/E60K/K189D as well as the wild-type Trypsin in correlation to various peptide substrates. Reaction conditions: 100 μM acyl donor (Bz PGGXaaXaaXaaHAG-OH), 200 μM acyl acceptor (H-XaaXaaXaaAK(DNP)-OH), 10 μM trypsin variant A2C8_H39Y/H59Y/E60K/K189D or 2.5 nM anionic rat trypsin II, 100 mM HEPES/NaOH pH 7.8, 0.1 mM ZnCl₂, 100 mM NaCl, 10 mM CaCl₂, T=30° C. UPLC analysis: Waters Acquity Ultra Performance LC, C18-column, gradient 5-60% acetonitrile, 5 min, detection at 360 nm.

FIG. 6: Dual modification of a Fab-fragment by utilizing variation in substrate specificity of two Trypsiligase II variants. The HER2-specific Fab-fragment of Trastuzumab (anti-Her2-Fab-LC_RRKH/HC_YRAH, Heavy chain (SEQ ID NO: 2) and anti-Her2-Fab-LC_RRKH/HC_YRAH, Light chain (SEQ ID NO: 3)) was used and the respective recognition sequences genetically introduced. A) Trypsiligase II variant K7F11_H39Y/H59Y/K189D which recognizes the sequence RRKH was used in the first step for attachment of a carboxyfluorescein(CF)-bearing nucleophile. B) In the second step Trypsiligase II variant A2C8 which recognizes the sequence YRAH was used for modification with a nucleophile covalently bound to mertansine (DM1). Analysis by LC-MS. The amount of modified and unmodified Fab-fragment species for the first and second modification step are shown in MS spectrum A and B, respectively. MS-spectrum A) Peak 1: anti-Her2-Fab-LC_R-OH/HC_YRAH (M_{calc.}=50116 Da, M_{found}=50118 Da), Peak 2: anti-Her2-Fab-LC_RRKH/HC_YRAH (M_{calc.}=50666 Da, M_{found}=50666 Da), Peak 3: anti-Her2-Fab-LC_RRKHAK(CF)/HC_YRAH (M_{calc.}=51095 Da, M_{found}=51097 Da). MS-spectrum B) Peak 1: anti-Her2-Fab-LC_RRKHAK(CF)/HC_Y-OH (M_{calc.}=49417 Da, M_{found}=49417 Da), Peak 2: anti-Her2-Fab-LC_R-OH/HC_YRKKAK(MCC-DM1) (M_{calc.}=50006 Da, M_{found}=50000 Da), Peak 3: anti-Her2-Fab-LC_RRKH/HC_YRKKAK(MCC-DM1) (M_{calc.}=50555 Da, M_{found}=50556 Da), Peak 4: anti-Her2-Fab-LC_RRKHAK(CF)/HC_YRKKAK(MCC-DM1) (M_{calc.}=50985 Da, M_{found}=50987 Da), Peak 5: anti-Her2-Fab-LC_RRKHAK(CF)/HC_YRAH (M_{calc.}=51095 Da, M_{found}=51095 Da). Reaction conditions first step: 100 μM Fab; 2000 μM RKHAK(CF)-OH; 5 μM K7F11_H39Y/H59Y/K189D; 100 mM HEPES/NaOH pH7.8, 100 mM NaCl, 10 mM CaCl₂, T=30° C., t=180 min. Reaction conditions second step: 50 μM Fab; 1000 μM RKKAK(MCC-DM1)-OH; 5 μM A2C8; 100 mM HEPES/NaOH pH7.8, 100 mM NaCl, 10 mM CaCl₂, T=30° C., t=40 min.

Methods and Materials

UPLC Analysis

Peptide and reaction analytics were performed using a Waters ACQUITY UPLC System equipped with an RP-C18 column (ACQUITY UPLC BEH 130, C18, 1.7 μm, 2.1×100 mm) at flow rates of 0.5 ml/min. The used mobile phases are: water with 0.05% TFA (A) and acetonitrile with 0.05% TFA (B), respectively. For analysis two methods were applied: Method I: linear gradient of 5-40% B in 5 min, detection at 254 nm.

Method II: linear gradient of 5-60% B in 5 min, detection at 360 nm.

Quantities of product and educt were calculated from the integrated peak areas.

Mass Spectrometry Analysis

Mass spectrometry (MS) analytics were performed by LC-MS with a Waters HPLC System connected to a Waters Micromass® ZQ™ MS-detector. LC separation was performed using a RP-C8 column (XBridge™, C8, 3.5 pM, 2.1×100 mm) at a flow rate of 0.3 ml/min. The used mobile phases are: water with 0.1% TFA (A) and acetonitrile with 0.1% TFA (B), respectively. For separation a linear gradient of 5-95% B in 10 min was applied with detection at 220 nm.

Expression and Purification of Trypsin Variants For recombinant production of all described trypsin variants the corresponding genes were subcloned into the pPICZαA expression vector using AgeI/XhoI restriction sites. E. coli DH5α was transformed with gene encoding vectors and the cells were subsequently plated on LB low salt (5 g/l yeast extract; 10 g/l tryptone; 5 g/l NaCl) agar plates containing 25 μg/ml Zeocin. After overnight incubation at 37° C. a single colony was picked and transferred into liquid LB low salt media containing 25 μg/ml Zeocin. The cells were incubated overnight at 37° C. under continuous shaking. The cells were harvested by centrifugation at 5000×g for 5 min followed by a plasmid isolation according to standard protocols. The isolated plasmids were linearized by SacI digestion. Following P. pastoris X-33 cells were transformed with the linearized plasmids by electroporation and plated on YPDS (10 g/l yeast extract; 20 g/l peptone; 20 g/l dextrose; 1 M sorbitol) agar plates containing 100 μg/ml Zeocin. These plates were incubated at 30° C. for three days. For expression of the trypsin variants a single colony was picked and transferred into buffered minimal media (100 mM potassium phosphate pH 6.0; 1.34% yeast nitrogen base) with 2% dextrose. After incubation for 48 h at 30° C. and continuous shaking cells were harvested at 4000×g for 5 min. Afterwards the cell pellet was resuspended in buffered minimal media and the protein production was induced by the addition of 1% (v/v) methanol, whereas the trypsin variants were secreted into the supernatant. The protein production was carried out by incubation for five days at 30° C. under continuous shaking with a daily addition of 1% (v/v) methanol. After five days the cells were separated from the supernatant by centrifugation at 5000×g for 20 min. For isolation of the secreted trypsin variants a two step purification was performed consisting of a cation exchange chromatography followed by a size exclusion chromatography. Using an AKTA FPLC a 20 ml HiPrep™ SP FF column (GE Healthcare) was equilibrated with 10 column volumes of binding buffer (20 mM sodium acetate pH 4.0). The supernatant was diluted with 1 volume binding buffer and loaded onto the column. After a washing step with 10 column volumes of binding buffer proteins were eluted with elution buffer (100 mM HEPES/NaOH pH 7.8, 200 mM NaCl, 10 mM CaCl₂) and protein containing fractions were detected by absorption at 280 nm. Pooled protein containing fractions were concentrated to a volume of about 1 ml using an Amicon® centrifugal filter device (NMWL: 10 kDa, Millipore). Subsequently the concentrated protein solution was purified by size exclusion chromatography using a HiLoad™ 16/60 Superdex™ 75 pg column (GE Healthcare) equilibrated with buffer (100 mM HEPES/NaOH pH 7.8, 100 mM NaCl, 10 mM CaCl₂). Protein containing fractions related to the absorption peak at 280 nm of the monomeric enzyme species (about 24 kDa) were identified by SDS-PAGE. Fractions having a purity >90% were pooled and concentrated with an Amicon® centrifugal filter device (NMWL: 10 kDa, Millipore). Protein concentration was determined by the absorption at 280 nm and the corresponding extinction coefficient of the variant. Identity of the trypsin variant was confirmed by LC-MS.

Expression and Purification of Fab-Fragment

For recombinant production of the Her2-specific Fab-fragment anti-Her2-Fab-LC_RRKH/HC_YRAH, the corresponding gene sequence (Seq. ID No. 64) was subcloned into the pASK-IBA7Plus expression vector by standard methods. E. coli BL21 (DE3) was transformed with the expression plasmid and plated on LB (5 g/l yeast extract; 10 g/l tryptone; 10 g/l NaCl) agar plates containing 100 μg/ml Ampicillin. After overnight incubation at 37° C. a single colony was picked and transferred into liquid LB media containing 100 μg/ml Ampicillin. For expression of the Fab-fragment this pre-culture was incubated overnight at 37° C. under continuous shaking and afterwards used for inoculation of the main culture in LB media containing 100 μg/ml Ampicillin (starting ODeoo nm of 0.1). When the culture reached an OD_{600 nm} of 0.8-1 protein production was induced by addition of 0.2 μg/ml anhydrotetracycline. After incubation for 4 h at 30° C. under continuous shaking the cells were harvested by centrifugation at 5000×g for 20 min. The pellet was resuspended in lysis buffer (20 mM sodium phosphate pH 7.0, 0.1 mM AEBSF). The cells were disrupted by sonication (8×10 sec, 30% Amplitude) and cell debris was removed by ultracentrifugation at 20000×g for 35 min. For isolation of the Fab-fragment a two step purification was performed consisting of a Protein G affinity chromatography followed by a size exclusion chromatography. Using an AKTA FPLC a 1 ml HiTrap™ Protein G HP column (GE Healthcare) was equilibrated with 10 column volumes of binding buffer (20 mM sodium phosphate pH 7.0). After loading the supernatant on the column a washing step was performed with 10 column volumes of binding buffer. Elution was carried out with 10 column volumes elution buffer (100 glycin/HCl pH 2.7) whereas the collected fractions were immediately neutralized with 20% (v/v) neutralization buffer (1 M Tris pH 9.0). Protein containing fractions were detected by absorption at 280 nm. Pooled protein containing fractions were concentrated to a volume of about 1 ml using an Amicon® centrifugal filter device (NMWL: 10 kDa, Millipore). Subsequently the concentrated protein solution was purified by size exclusion chromatography using a HiLoad™ 16/60 Superdex™ 75 pg column (GE Healthcare) equilibrated with buffer (100 mM HEPES/NaOH pH 7.8, 100 mM NaCl, 10 mM CaCl₂). Fractions related to the absorption peak at 280 nm of the monomeric Fab species (about 50 kDa) were identified by SDS-PAGE. Fractions having a purity >90% were pooled and concentrated with an Amicon® centrifugal filter device (NMWL: 10 kDa, Millipore). The final protein concentration was determined by the absorption at 280 nm and the corresponding extinction coefficient. Identity of Fab-fragment was confirmed by LC-MS.

Peptide Synthesis

All reagents and detergents for peptide synthesis were purchased in the highest commercially available quality at Sigma Aldrich®, Germany. All amino acids as well as building blocks (Lys(Dnp), Lys(ivDDE)) for peptide synthesis were purchased at IRIS® Biotech GmbH, Marktredwitz, Germany. DM1 was purchased at Concortis®, San Diego, USA.

The peptides were synthesized by standard procedures using an Fmoc/protecting group strategy as described by Merrifield. According to the final peptide sequence the first amino acid was coupled to a chlorotrityl resin. In the following Fmoc cleavage was performed by using 20% piperidine in DMF. For further coupling, the amino acids were activated using ((1-[bis(dimethylamino)methylene]-1H-1,2,3-triazolo[4,5-b]pyridinium 3-oxide hexafluorophosphate) (HATU). The final liberation from the resin and deprotection of the side chain protecting groups was done by using 95% TFA, 2.5% triisopropylsilane and 2.5% water. After vaporizing the solvent, the oily remainder was dissolved in water/ACN and purified via preparative HPLC (Merck/Hitachi-HPLC, Vydac-C18, 5 to 80% ACN, 30/60 min). After freeze drying the product containing fractions of the peptides were obtained as a crystalline powder. Product identity and purity was proven by HPLC (Waters, ACQUITY UPLC, BEH130) and LC-MS (Waters, X-Bridge BEH300). The purity of all peptides was higher than 98%.

For synthesizing the mertansine (DM1) functionalized peptide H-RKKAK(MCC-DM1)-OH the building block Fmoc-Lys(ivDde) was used. After synthesis of the RKKAK(ivDde) peptide the protecting group of the lysine was removed from the fully protected peptide by using 2% Hydrazine in DMF. Afterwards the lysine side chain was functionalized with 1.1 eq. Succinimidyl 4-(N-maleimidomethyl)cyclohexane-1-carboxylate in DMF. In a final step the RKKAK(MCC) was coupled via a Michael addition to Mertansin using a buffered phosphate buffer/ACN system (pH 7.4)

The final liberation from the resin and deprotection of the side chain protecting groups as well as the purification of H-RKKAK(MCC-DM1)-H was done as described before. The product was obtained as an isomeric mixture of two species. The product identity and purity was proven by UPLC and LC-MS. The purity of H-RKKAK(MCC-DM1)-OH was higher than 99%.

Model Transamidation Reaction Catalyzed by Trypsiliqase II and Improved Trypsiliqase II Variants

Model transamidation reactions were performed in a solution containing 15 to 250 μM acyl donor, 2 eq. of the corresponding acyl acceptor, varying concentrations of trypsin variant, 100 mM HEPES/NaOH pH 7.8, ±0.1 mM ZnC12, 100 mM NaCl, 10 mM CaCl₂) at 30° C. Acyl donor and acyl acceptor had the same amino acids in P₁′-P₃′ position, e.g. Bz-PGGYRAHAG+H-RAHAK-OH or Bz-PGGYRKKAG+H-RKKAK-OH. Variations are indicated. The acyl acceptor could be equipped with a 2,4-Dinitrophenyl group (DNP) at the side chain of the terminal lysine, e.g. H-RAHAK(DNP)-OH or H-RKKAK(DNP)-OH. The reactions were started by the addition of enzyme. To record the time course of product formation and/or for kinetic evaluation of the reaction several aliquots of the reaction mixture were quenched with 25% (v/v) acetic acid at distinct time points within up to 4 hours. The composition of the reaction mixture was analyzed by UPCL with method I or II, depending on the absence (method 1) or presence (method II) of a 2,4-dinitrophenyl group within the acyl acceptor (see example 1). Measurements were performed as duplicates and errors were less than 5%.

Determination of Turnover Rates for the Hydrolysis Reaction Catalyzed by Trypsiliqase II and Improved Trypsiliqase II Variants

Hydrolysis reactions were performed in a solution containing varying concentrations of the acyl donor Bz-PGGYRAHAG-OH (0-5000 μM for Trypsiligase II, 0-1500 μM for variant A2C8), 2 μM trypsin variant, 100 mM HEPES/NaOH pH 7.8, 100 mM NaCl, 10 mM CaCl₂ at 30° C. In case of Trypsiligase II the reactions were performed in the presence of 100 μM ZnCl₂. The reaction was started by the addition of enzyme. For kinetic evaluation of the reaction several aliquots of the reaction mixture were quenched with 25% (v/v) acetic acid at distinct time points within 30 minutes. The composition of the reaction mixture was analyzed by UPCL using method I (see example 1). UV absorption of the N-terminal benzoyl-group (Bz-), which is present in the acyl donor as well as in the hydrolysis product, was detected at 254 nm. To determine the turnover rate of the hydrolysis reaction (k_{cat,HL}) the initial rates for the hydrolysis reaction were plotted against the corresponding acyl donor concentration and fitted to the Michaelis Menten equation. Measurements were performed as duplicates and errors were less than 5%.

Determination of the K_{M} Values of the Nucleophilic Peptide

For Trypsiligase II the measurements for K_{M} values of the nucleophilic peptide (acyl acceptor) were carried out in the presence or absence of zinc ions due to its strong zinc ion dependency. In case of variant A2C8 measurements were carried out in the absence of zinc ions.

Transamidation reactions with Trypsiligase II and variant A2C8 were performed in a solution containing 250 μM of the acyl donor Bz-PGGYRAHAG-OH, varying concentrations of the acyl acceptor H-RAHAK(DNP)-OH (0-5000 μM for Trypsiligase II in the absence of zinc ions, 0-1500 μM for Trypsiligase II in the presence of zinc ions, 0-1000 μM for variant A2C8), 0.2-1.5 μM trypsin variant, 100 mM HEPES/NaOH pH 7.8, ±0.1 mM ZnCl₂, 100 mM NaCl, 10 mM CaCl₂ at 30° C. The reaction was started by the addition of enzyme. For kinetic evaluation of the reaction several aliquots of the reaction mixture were quenched with 25% (v/v) acetic acid at distinct time points within 30 minutes. The composition of the reaction mixture was analyzed by UPCL using method II (see example 1). UV absorption of the 2,4-Dinitrophenyl group (DNP), which is present in the acyl acceptor as well as in the aminolysis product, was detected at 360 nm. To determine the K_{M} value of the nucleophilic peptide the initial rates for the aminolysis reaction were plotted against the corresponding acyl acceptor concentration and fitted to the Michaelis Menten equation. Measurements were performed as duplicates and errors were less than 5%.

Orthogonal Dual-Modification of a Her2-Specific Fab-Fragment

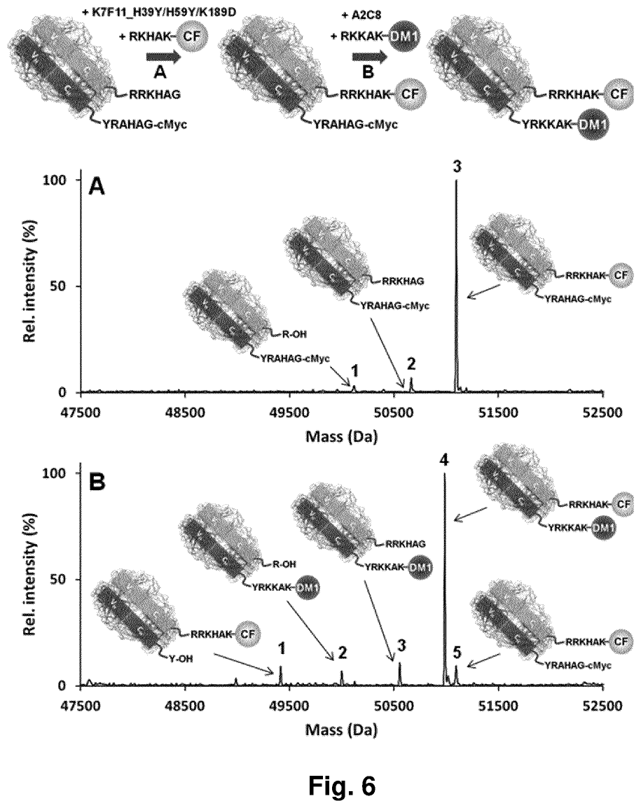

For the dual-modification of a Her2-specific Fab-fragment, two orthogonal recognition sequences were attached to the corresponding C-terminal end of the heavy and light chain, respectively. This was done to enable the enzyme mediated conjugation of two different functionalities. As depicted in Seq. ID No. 2, the light chain was elongated at the C-terminal end by a short peptide-spacer (LSPGG), followed by the amino acid sequence RRKHAG, which contains the recognition sequence of variant K7F11_H39Y/H59Y/K189D (RRKH). The C-terminal end of the heavy chain was elongated by a short peptide-spacer (ADKPGG), followed by the amino acid sequence YRAHAG, which contains the recognition sequence of variant A2C8 (YRAH) and a cMyc-tag (EQKLISEEDL) for optional purification or detection purposes. The dual-modification of the Her2-specific Fab-fragment containing the two orthogonal recognition sequences (aHer2-Fab-LC_RRKH-HC_YRAH) was performed by a two-step modification reaction, attaching the fluorescent dye (5(6)-Carboxyfluorescein) at the light chain in a first step and the cytotoxic compound Mertansine (DM1) at the heavy chain in a second step (FIG. 6).

The transamidation reaction for the modification of the light chain was performed in a solution containing 100 μM aHer2-Fab-LC_RRKH/HC_YRAH, 2000 μM H-RKHAK(CF)-OH, 5 μM K7F11_H39Y/H59Y/K189D, 100 mM HEPES/NaOH pH 7.8, 100 mM NaCl. The reaction was started by the addition of enzyme and incubated at 30° C. for 180 min. Subsequently the enzyme as well as the remaining Carboxyfluorescein bearing nucleophile (H-RKHAK(CF)-OH) were removed by a Protein G affinity chromatography. Using an AKTA FPLC a 1 ml HiTrap™ Protein G HP column (GE Healthcare) was equilibrated with 10 column volumes of binding buffer (20 mM sodium phosphate pH 7.0). After applying the reaction mixture, the column was washed with 10 column volumes of binding buffer. Following bound Fab-fragment species were eluted with 10 column volumes elution buffer (100 glycin/HCl pH 2.7), whereas the collected fractions were immediately neutralized with ⅕th volume neutralization buffer (1 M Tris pH 9.0). Protein containing fractions were pooled and the buffer was exchanged to 100 mM HEPES/NaOH pH 7.8, 100 mM NaCl using a Amicon® centrifugal filter device (NMWL: 10 kDa, Millipore).

The second modification reaction of the heavy chain was performed in a solution containing 50 μM of the single modified aHer2-Fab-LC_RRKHAK(CF)/HC_YRAH, 1000 μM H-RKKAK(MCC-DM1)-OH, 5 μM A2C8, 100 mM HEPES/NaOH pH 7.8, 100 mM NaCl. The reactions was started by the addition of enzyme and incubated at 30° C. for 40 min. Subsequently the enzyme as well as the remaining DM1 bearing nucleophile (H-RKHAK(MCC-DM1)-OH) were removed by a Protein G affinity chromatography as described above. The proportions of modified and unmodified Fab-fragment species for the first and second modification step were analyzed by LC-MS (see example 1). After the first modification step up to 90% of the Fab-fragment was exclusively modified with Carboxyfluorescein at the light chain (anti-Her2-Fab-LC_RRKHAK(CF)/HC_YRAH, FIG. 6 spectrum A Peak 3, M_{calc.}=51095 Da. M_{found}=51097 Da). In addition two minor side products could be identified corresponding to the unconsumed Fab-Fragment (anti-Her2-Fab-LC_RRKH/HC_YRAH, FIG. 6 spectrum A Peak 2, M_{calc.}=50666 Da, M_{found}=50666 Da) as well as to a Fab-Fragment with a hydrolyzed recognition sequence at the light chain (anti-Her2-Fab-LC_R-OH/HC_YRAH, FIG. 6 spectrum A Peak 1, M_{calc.}=50116 Da, M_{found}=50118 Da). No modification upon the recognition sequence at the heavy chain had been monitored.

After the second modification step up to 75% of the Fab-Fragment was dual modified with Carboxyfluorescein at the light chain and DM1 at the heavy chain (anti-Her2-Fab-LC_RRKHAK(CF)/HC_YRKKAK(MCC-DM1), FIG. 6 spectrum B Peak 4, M_{calc.}=50985 Da, M_{found}=50987 Da) representing the desired configuration. In addition four minor side products were detected corresponding to the single modified Fab-Fragment with Carboxyfluorescein at the light chain and full length recognition sequence at the heavy chain (anti-Her2-Fab-LC_RRKHAK(CF)/HC_YRAH, FIG. 6 spectrum B Peak 5, M_{calc.}=51095 Da, M_{found}=51095 Da), single modified Fab-Fragment with DM1 at the heavy chain and full length recognition sequence at the light chain (anti-Her2-Fab-LC_RRKH/HC_YRKKAK(MCC-DM1), FIG. 6 spectrum B Peak 3, M_{calc.}=50555 Da, M_{found}=50556 Da), single modified Fab-Fragment with DM1 at the heavy chain and hydrolyzed recognition sequence at the light chain (anti-Her2-Fab-LC_R-OH/HC_YRKKAK(MCC-DM1), FIG. 6 spectrum B Peak 2, M_{calc.}=50006 Da, M_{found}=50000 Da) as well as single modified Fab-Fragment with Carboxyfluorescein at the light chain and hydrolyzed recognition sequence at the heavy chain (anti-Her2-Fab-LC_RRKHAK(CF)/HC_Y-OH, FIG. 6 spectrum B Peak 1, M_{calc.}=49417 Da, M_{found}=49417 Da). Again no modification at the C-terminal end of the light chain was detected, which could be related to variant A2C8.

### EXAMPLES

### Example 1

The measurements of the product yield and the apparent tumover rates for aminolysis (k_{cat,AL}^{app}) and hydrolysis (k_{cat,HL}^{app}) were performed by a model transamidation reaction with the following conditions: 250 μM Bz-AAYRHAAG-OH (acyl donor), 500 μM H-RHAK-OH (acyl acceptor), 5-10 μM trypsin variant, 100 mM HEPES/NaOH pH 7.8, 0.1 mM ZnCl₂, 100 mM NaCl, 10 mM CaCl₂, T=30° C. K_{M} values for the acyl acceptor were determined by the measurements of apparent turnover rates for aminolysis at constant acyl donor concentration and varying acyl acceptor concentrations.

TABLE 1
Summary for enzymatic parameters for Trypsiligase
I as well as the improved variants 2G10 and 1C11.

| Variant | product yield (%) | $\begin{matrix}{{k}_{cat , AL}^{app}}_{\underline{\_}} \\ ( mkat \text{/} mol )\end{matrix}$ | k_{cat,HL}^{app} (mkat/mol) | k_{cat,AL}^{app}/k_{cat,HL}^{app} | K_{M}, acyl acceptor (μM) |
|---|---|---|---|---|---|
| Trypsiligase I | 42.2 | 47.1 | 11.1 | 4.2 | 222 |
| 2G10 | 59.3 | 65.1 | 2.5 | 26.0 | 23 |
| 1C11 | 60.9 | 16.0 | 0.3 | 57.0 | n.d. |
| | | | | | (>5000) |

2G10 shows an increased affinity for the acyl acceptor (23 μM) in comparison to native Trypsiligase (222 μM), and 1C11 has a decreased affinity for the acyl acceptor (>5000 μM). Both improved variants possess a significant increase for the ratio of aminolysis to hydrolysis activity (factor 6 and 13 for 2G10 and 1C11). This correlates with an improved synthesis efficiency reflected by the increased product yield. For 2G10 the improved aminolysis to hydrolysis ratio is a result of the better affinity for the peptidic nucleophile. This comes along with a direct competition between a water molecule and the peptide for a nucleophilic attack on the acyl-enzyme-intermediate in the deacylation step of the transamidation reaction. For 1C11 the improved aminolysis to hydrolysis ratio is a result of a significantly reduced hydrolysis activity (factor 37) whereas the aminolysis activity is lowered by factor 3. The mutations of 2G10 and 1C11 were combined in one variant (hybrid) to check, whether there is a synergistic effect for the synthesis efficiency due to different modes of improvement.

### Example 2

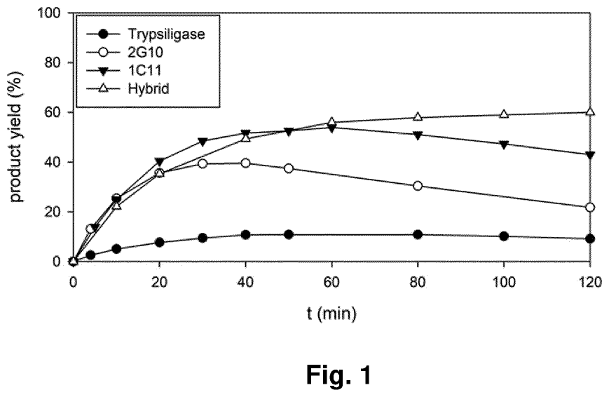

On the basis of these enzyme kinetic observations, the positions identified in these two variants 2G10 and 1C11 have been combined in a hybrid variant. As illustrated in Table 2 and FIG. 1 the hybrid variant resulting from the combination of the positions identified in the variants 2G10 and 1C11 benefits from both positive effects.

The measurements of the product yield and the apparent tumover rates for aminolysis (k_{cat,AL}^{app}) and hydrolysis (k_{cat,HL}^{app}) were performed by a model transamidation reaction with the following conditions: 15 μM Bz-AAYRHAAG-OH (acyl donor), 30 μM H-RHAK-OH (acyl acceptor), 0.5-2.1 μM trypsin variant, 100 mM HEPES/NaOH pH 7.8, 0.1 mM ZnCl₂, 100 mM NaCl, 10 mM CaCl₂, T=30° C.

TABLE 2
Summary of enzymatic parameters for Trypsiligase
I, 2G10, 1011 and the hybrid variant.

| | product yield | k_{cat, AL}^{app} | k_{cat, HL}^{app} | k_{cat, AL}^{app}/ |
|---|---|---|---|---|
| Variant | (%) | (mkat/mol) | (mkat/mol) | k_{cat, HL}^{app} |
| Trypsiligase I | 10.9 | 2.36 | 5.84 | 0.4 |
| 2G10 | 39.6 | 16.39 | 5.03 | 3.3 |
| 1C11 | 53.9 | 6.24 | 0.97 | 6.4 |
| Hybrid | 60 | 2.11 | 0.05 | 42.2 |

The hybrid variant shows a further increase in synthesis efficiency which is reflected by a improved product yield as a result of a significantly better aminolysis to hydrolysis ratio, especially at low substrate concentrations (15 μM). This leads to the conclusion that there is a synergistic effect resulting a trypsin variant that shows better synthesis properties than the native Trypsiligase I as well as the improved variants 2G10 and 1C11. To generate a new trypsin variant with alternated recognition sequence, a new trypsin library was designed, based on Trypsiligase II, including amino acid positions that were shown to improve synthesis efficiency of Trypsiligase I. This library was subjected to a selection via phage display for an enrichment of potentially improved transamidases using two different substrates with the recognition sequence YRAH and YRKH.

### Example 3

On the basis of these investigations the mutated positions of these two variants 2G10 and 1C11 have been combined in a new enzyme library based on trypsin variant Y39H/Y59H/K60E/D189K (Trypsiligase II), so that the variants resulting from this library benefit from both positive effects and are thus further optimized. Accordingly, positions 40, 55, 96, 97, 143, 151, 190, 192, 214, 219 and 221 were randomized in this Trypsiligase II library, while the mutation L99F was fixed because it was present in both Trypsiligase I libraries A and B. Positions 143 and 151 in Trypsiligase I are responsible for zinc complexation and thus convey histidine specificity for the recognition sequence YRH. In Trypsiligase II, this histidine specificity is shifted by positions 39 and 59, resulting in the recognition sequence YRAH. Thus, positions 143 and 151, which are potentially responsible for P₂′ position specificity, are available for randomization in the Trypsiligase II library. In addition to the desired effect of increasing the affinity to the substrate/nucleophile, this could also lead to a possible independence of zinc complexation, which would be desirable with regard to the application-oriented modification of recombinant proteins. Using phage display and an ELISA-based screening, the Trypsiligase II variants illustrated in Table 3 and 4 have been identified.

TABLE 3
Summary of variants identified during phage-display selection and screening out of
Trypsiligase II-library selected with a YRAH-containing substrate and related data
regarding enzymatic activity and maximum product yield. 100 μM Bz-PGGYR(A/K)HAG-
OH; 200 μM R(A/K)HAK-OH; 100 mM HEPES pH 7.8, 100 mM NaCl, 10 mM CaCl₂,
100 μM ZnCl₂, T = 30° C.; 4Tn II = Trypsiligase II; Pₘₐₓ = maximum yield in %; A_{S}[AL] =
specific aminolysis activity; A_{S}[HL] = specific hydrolysis activity; A_{S} A/H = quotient of aminolysis/
hydrolysis activity; YRAH/YRKH = quotient of max. product yield for YRAH subtrate/YRKH substrate.

| | Mutation | | | | | | | | | | |
|---|---|---|---|---|---|---|---|---|---|---|---|
| Variant | P40 | P55 | P96 | P97 | P143 | P151 | P190 | P192 | P214 | P219 | P221 |
| A2C8 | F | A | E | D | E | Y | V | A | G | Q | T |
| 1G4 | M | S | P | F | T | E | A | N | G | L | D |
| A9D5 | H | T | P | H | T | A | S | F | G | W | T |
| A3C7 | F | A | E | F | F | D | A | W | G | S | A |
| A7B9 | Y | A | P | H | E | T | T | F | G | E | Q |
| A6D8 | F | A | E | F | R | K | V | S | G | G | M |
| A5C7 | H | A | P | H | E | P | V | F | G | W | D |
| A8F12 | M | A | E | F | M | N | V | N | G | E | Q |
| A8D1, A6F2 | F | A | E | H | D | S | S | W | S | A | P |
| ASA3 | F | A | E | F | F | D | A | W | S | R | E |
| A6F9 | W | A | E | F | T | A | A | Y | A | W | P |
| A8G7 | L | A | V | D | F | D | A | W | G | S | A |
| A7E11 | T | A | E | H | I | D | S | Y | S | R | E |
| 4Tn II | H | A | R | K | N | E | S | Q | S | G | A |
| A6B12 | F | A | P | H | S | N | A | N | S | Q | H |
| A8A8, A8D11 | M | A | V | D | N | E | S | A | S | R | E |

| | Pₘₐₓ | | | | PPₘₐₓ | | | | | |
|---|---|---|---|---|---|---|---|---|---|---|
| | _{vs YRAH} | A_{S}[AL] | A_{S}[HL] | A_{S} | _{vs YRAH} | YRAH/ | A_{S}[AL] | A_{S}[HL] | A_{S} | A_{S} A[YRAH]/AS |
| Variant | _{(%)} | (mkat/mol) | (mkat/mol) | A/H | (%) | YRKH | (mkat/moL) | (mkat/moL) | A/H | A + H[YRAH] |
| A2C8 | 53.1 | 15.8 | 1.5 | 10.9 | 51.3 | 1.0 | 8.9 | 1.3 | 6.9 | 1.6 |
| 1G4 | 47.6 | 2.2 | 0.3 | 7.6 | 36.8 | 1.3 | 0.8 | 0.2 | 3.5 | 2.3 |
| A9D5 | 45.9 | 1.2 | 0.1 | 9.0 | 15.5 | 3.0 | 0.2 | 0.1 | 1.6 | 3.5 |
| A5C7 | 41.6 | 1.9 | 0.4 | 4.6 | — | — | — | — | — | — |
| A7B9 | 37.2 | 2.9 | 0.5 | 4.3 | 31.2 | 1.2 | 0.7 | 0.4 | 1.5 | 1.8 |
| A6D8 | 30.6 | 1.8 | 1.0 | 1.8 | 7.4 | 4.1 | 0.2 | 0.3 | 0.5 | 3.6 |
| ASC7 | 30.3 | 1.8 | 0.8 | 2.1 | 22.9 | 1.3 | 0.5 | 0.6 | 9.8 | 1.7 |
| A8F12 | 20.2 | 2.2 | 2.3 | 1.0 | — | — | — | — | — | — |
| A8D1, A6F2 | 14.0 | 11.0 | 13.1 | 0.8 | 29.6 | 0.5 | 24.6 | 30.3 | 0.8 | 0.2 |
| A8A3 | 13.2 | 1.6 | 3.4 | 0.5 | 21.1 | 0.6 | 3.5 | 5.6 | 0.6 | 0.2 |
| A6F9 | 13.0 | 1.9 | 5.5 | 0.4 | 10.3 | 1.3 | 0.6 | 1.5 | 0.4 | 0.9 |
| A8G7 | 12.3 | 0.5 | 0.2 | 3.3 | 18.4 | 0.7 | 0.6 | 0.7 | 9.9 | 0.4 |
| A7E11 | 10.0 | 18.2 | 72.6 | 0.3 | 17.1 | 0.6 | 28.5 | 84.1 | 0.3 | 0.2 |
| 4Tn II | 8.9 | 2.4 | 7.8 | 0.3 | 1.5 | 6.0 | 0.5 | 16.3 | 0.0 | 0.1 |
| A6B12 | 7.5 | 1.3 | 7.7 | 0.2 | 3.6 | 2.1 | 0.4 | 4.9 | 0.1 | 0.2 |
| A8A8, A8D11 | 6.8 | 1.6 | 2.6 | 0.6 | 9.4 | 0.7 | 0.4 | 1.5 | 0.3 | 0.8 |

TABLE 4
Summary of variants identified during phage-display selection and screening out of
Trypsiligase II-library selected with a YRKH-containing substrate and related data
regarding enzymatic activity and maximum product yield. 100 μM Bz-PGGYR(A/K)HAG-
OH; 200 μM R(A/K)HAK-OH; 100 mM HEPES pH 7.8, 100 mM NaCl, 10 mM CaCl₂,
100 μM ZnCl₂, T = 30° C.; 4Tn II = Trypsiligase II; Pₘₐₓ = maximum yield in %; A_{S}[AL] =
specific aminolysis activity; A_{S} [HL] = specific hydrolysis activity; A_{S} A/H = quotient of aminolysis/
hydrolysis activity; YRAH/YRKH = quotient of max. product yield for VRAH subtrate/YRKH substrate.

| | Mutation | | | | | | | | | | |
|---|---|---|---|---|---|---|---|---|---|---|---|
| Variant | P40 | P55 | P96 | P97 | P143 | P151 | P190 | P192 | P214 | P219 | P221 |
| K5E10 | Y | A | E | F | V | D | V | D | G | P | Q |
| K7F11 | Y | A | E | F | V | E | A | V | G | P | Q |
| K1A4* | Υ | A | E | F | V | D | A | V | G | P | Q |
| K7G12 | Υ | A | E | F | V | D | A | V | G | P | T |
| K4H1 | S | A | P | H | D | T | A | P | G | P | H |
| K6F12 | Υ | A | S | H | V | D | A | V | G | P | Q |
| K5G6 | V | A | E | F | V | D | A | V | G | P | Q |
| K1G9 | Υ | A | E | F | V | G | A | V | G | P | Q |
| K5E6 | W | A | S | H | E | D | A | Y | S | P | H |
| K3G5 | N | S | P | H | D | T | A | W | S | W | P |
| K3B11 | Υ | A | V | H | T | W | S | N | S | S | Q |
| 4Tn II | H | A | R | K | N | E | S | Q | S | G | A |

| | PPₘₐₓ | | | | PPₘₐₓ | | | | | |
|---|---|---|---|---|---|---|---|---|---|---|
| | _{vs YRAH} | A_{S}[AL] | A_{S}[HL] | A_{S} | _{vs YRAH} | YRAH/ | A_{S}[AL] | A_{S}[HL] | A_{S} | A_{S} A[YRAH]/AS |
| Variant | _{(%)} | (mkat/mol) | (mkat/mol) | A/H | (%) | YRKH | (mkat/moL) | (mkat/moL) | A/H | A + H[YRAH] |
| K5E10 | 41.1 | 6.8 | 1.7 | 4.0 | 27.9 | 1.5 | 5.4 | 3.3 | 1.7 | 0.8 |
| K7F11 | 35.7 | 18.1 | 7.5 | 2.4 | 48.3 | 9.7 | 31.0 | 7.1 | 4.4 | 0.5 |
| K1A4* | 35.4 | 5.3 | 2.3 | 2.3 | 30.2 | 1.2 | 4.5 | 2.5 | 1.8 | 0.8 |
| K7G12 | 33.1 | 4.0 | 1.8 | 2.2 | 30.5 | 1.1 | 2.2 | 1.3 | 1.7 | 1.1 |
| K4H1 | 29.8 | 9.1 | 6.3 | 1.4 | 23.0 | 1.3 | 4.8 | 5.1 | 0.9 | 0.9 |
| K6F12 | 29.8 | 3.7 | 2.4 | 1.5 | 24.3 | 1.2 | 1.7 | 1.4 | 1.2 | 1.2 |
| K5G6 | 26.2 | 2.9 | 2.4 | 1.2 | 18.5 | 1.4 | 1.3 | 1.2 | 1.1 | 1.2 |
| K1G9 | 24.1 | 6.3 | 5.5 | 1.1 | 32.2 | 0.7 | 12.2 | 6.8 | 1.8 | 0.3 |
| K5E6 | 23.1 | 123.6 | 150.4 | 0.8 | 18.0 | 1.3 | 9S.4 | 171.2 | 0.6 | 0.5 |
| K3G5 | 10.9 | 0.2 | 0.5 | 0.4 | 5.6 | 1.9 | 0.1 | 0.2 | 0.4 | 0.6 |
| K3B11 | 9.0 | 1.5 | 7.6 | 0.2 | 11.7 | 0.8 | 1.9 | 7.3 | 0.3 | 0.2 |
| 4Tn II | 1.5 | 0.5 | 16.3 | 0.0 | 8.9 | 0.2 | 2.4 | 7.8 | 0.3 | 0.0 |

For the identification of improved biocatalysts the two variant pools of the 4^{th} round of selection via phage display were subjected to an ELISA-based high-throughput-screening. In total 26 trypsin variants could be identified that showed an increased synthesis efficiency ether with the YRAH substrate or the YRKH substrate. No variant was identified, which clearly discriminates between the two recognition sequences.

### Example 4

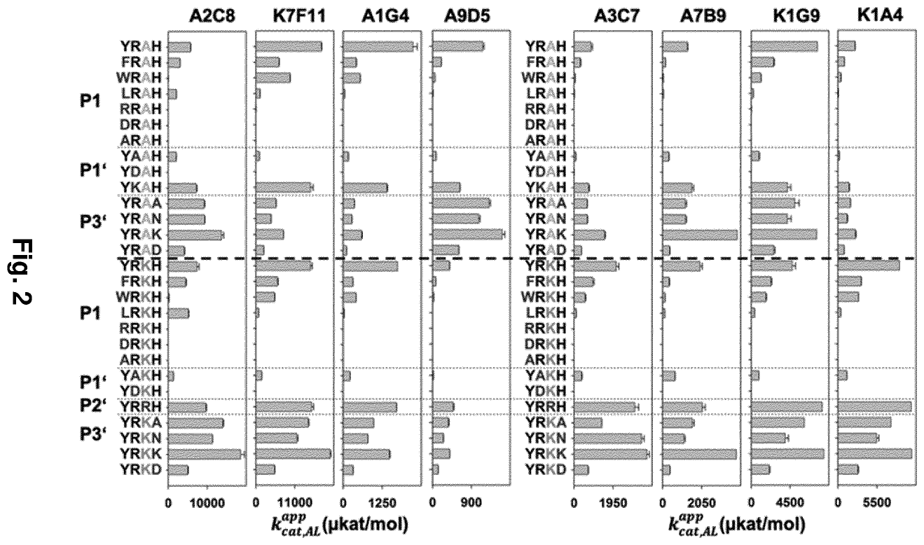

The most promising variants identified via phage-display selection were subjected to a further characterization concerning the substrate specificity as illustrated in FIG. 2.

Variants A2C8 and K7F11 showed the highest activity for YRKK substrate sequence. High flexibility in specificity for the P₃∝-position was observed for all variants. This leads to assumption that zinc-ions are redundant for optimized variants. In addition variant A2C8 and K7F11 could be potentially orthogonal biocatalysts as they possess an orthogonal pair of recognition sequences. A2C8 accepts LRKH as acyl donor which is not the case for K7F11. K7F11 accepts WRAH as acyl donor which is not the case for A2C8.

For the most promising variants A2C8 (Trypsiligase II+H40F, A55A, R96E, K97D, L99F, N143E, E151Y, S190V, Q192A, S214G, G219Q, A221T) and K7F11 (Trypsiligase II+H40Y, A55A, R96E, K97E, L99F, N143V, E151E, S190A, Q192V, S214G, G219P, A221Q) further studies concerning the dependence of metal ions as well as the synthesis efficiency were performed.

The results regarding zinc dependence of the transamidation reaction catalyzed by Trypsiligase II, A2C8 and K7F11 are illustrated in Table 5. The results of the kinetic measurements done for Trypsiligase II, and variants A2C8 and K7F11 are illustrated in Table 6.

TABLE 5
Study for the zinc dependence of the transamidation reaction
catalysed by Trypsiligase II, A2C8 and K7F11. The measurements
of the product yield and the apparent turnover rates for aminolysis
(k_{cat, AL}^{app}) and hydrolysis (k_{cat, HL}^{app}) were performed by a model
transamidation reaction in the presence or absence of zinc ions.
Reaction conditions for A2C8 and K7F11: 100 μM Bz-PGGYRXₐₐXₐₐAG-
OH (acyl donor), 200 μM H-RXₐₐXₐₐAK(DNP)-OH (acyl acceptor), 2 μM
trypsin variant, 100 mM HEPES/NaOH pH 7.8, ±0.1 mM ZnCl₂, 100 mM
NaCl, 10 mM CaCl₂, T = 30° C. Reaction conditions for Trypsiligase II:
250 μM Bz-AAYRAHAG-OH (acyl donor), 500 μM H-RAHAK(DNP)-
OH (acyl acceptor), 2 μM trypsin variant, 100 mM HEPES/NaOH
pH 7.8, ±0.1 mM ZnCl₂, 100 mM NaCl, 10 mM CaCl₂, T =
30° C. UPLC analysis: Waters Acquity Ultra Performance LC,
C18-column, gradient 5-60% acetonitrile, 5 min, detection at
360 nm; R₁: Bz-PGG, R₂: AG-OH

| | | | | product | product |
|---|---|---|---|---|---|
| | | k_{cat, AL}^{app} + | k_{cat, AL}^{app} − | yield + | yield − |
| | Substrate | Zn²⁺ | Zn²⁺ | Zn²⁺ | Zn²⁺ |
| Variant | sequence | (mkat/mol) | (mkat/mol) | (%) | (%) |
| Tryp- | R₁-YRAH- | 11.19 | 0.89 | 18.1 | 4.2 |
| siligase II | R₂ | | | | |
| A2C8 | R₁-YRAH- | 9.46 | 14.51 | 53.3 | 50.4 |
| | R₂ | | | | |
| | R₁-YRKH- | 8.42 | 16.92 | 55.0 | 57.6 |
| | R₂ | | | | |
| | R₁-YRKK- | 18.04 | 17.7 | 59.0 | 59.7 |
| | R₂ | | | | |
| K7F11 | R₁-YRAH- | 27.04 | 9.39 | 43.3 | 25.8 |
| | R₂ | | | | |
| | R₁-YRKH- | 17.85 | 23.82 | 41.9 | 42.3 |
| | R₂ | | | | |
| | R₁-YRKK- | 22.03 | 28.23 | 44.5 | 46.8 |
| | R₂ | | | | |

Native Trypsiligase II shows a strong dependence for zinc ions. In the absence of zinc ions the apparent turnover rate for the aminolysis reaction drops by a factor of 12 which results in a decrease of product yield from 18 to 4%. The reason for this is a lowered affinity for the nucleophile due to the missing complexation between the artificial histidines of Trypsiligase II (H39 and H59) and the peptide localized histidin with a zinc ion as central atom. A2C8 shows no dependence for zinc ions using the two substrates with YRAH and YRKH sequence, whereas the apparent turnover rate for the aminolysis reaction benefits from the absence of zinc ions. This allows the substitution of the histidine in P₃′ position as shown for YRKK substrate which additionally has an increasing effect on the turnover rate for the aminolysis reaction as well as for the product yield. K7F11 shows only for the YRAH substrate a slightly zinc ion dependence. In the absence of zinc ions the turnover rate for the aminolysis reaction drops by a factor of 2 which results in a decrease of product yield from 43 to 26%. This dependence could be abolished by the substitution of the alanine in P₂′-position against a lysine which leads to a better synthesis performance in the absence of zinc ions. Also K7F11 tolerates the substitution of the P₃′ histidine leading also to an increasing effect on the apparent turnover rate for the aminolysis reaction as well as for the product yield. The missing zinc dependence for K7F11 and A2C8 leads to the assumption, that the artificial histidins of Trypsiligase II could be back mutated to the native amino acids found in wild-type trypsin without influencing the favorable synthesis properties that were gained by the newly introduce mutations at the randomized positions.

TABLE 6
Summary of enzymatic parameters for Trypsiligase II, A2C8 and
K7F11. The measurements of the product yield and the apparent
turnover rates for aminolysis (k_{cat, AL}^{app}) and hydrolysis (k_{cat, HL}^{app})
were performed by a model transamidation reaction at high and low
substrate concentrations with two equivalents of the corresponding
nucleophile. Reaction conditions A2C8 and K7F11: 250/15 μM Bz-
PGGYRKKAG-OH (acyl donor), 500/30 μM H-RKKAK-OH (acyl
acceptor), 0.75-2 μM trypsin variant, 100 mM HEPES/NaOH pH 7.8, 100
mM NaCl, 10 mM CaCl₂, T = 30° C. Reaction conditions Trypsiligase
II: 250/15 μM Bz-PGGYRAHAG-OH (acyl donor), 500/30 μM H-
RAHAK-OH (acyl acceptor), 0.75-2.5 μM trypsin variant, 100 mM
HEPES/NaOH pH 7.8, 0.1 mM ZnCl₂, 100 mM NaCl, 10 mM CaCl₂, T =
30° C. UPLC analysis: Waters Acquity Ultra Performance LC, C18-
column, gradient 5-40% acetonitrile, 4 min, detection at 254 nm.

| | [Sub- | product | | | |
|---|---|---|---|---|---|
| | strate] | yield | k_{cat, AL}^{app} | k_{cat, HL}^{app} | k_{cat, AL}^{app}/ |
| Variant | (μM) | (%) | (mkat/mol) | (mkat/mol) | k_{cat, HL}^{app} |
| Trypsiligase | 250 | 17.3 | 14.40 | 16.00 | 0.9 |
| II | 15 | 3.2 | 0.33 | 2.84 | 0.1 |
| A2C8 | 250 | 64.9 | 71.61 | 1.01 | 70.9 |
| | 15 | 46.2 | 10.68 | 1.44 | 7.4 |
| K7F11 | 250 | 55.3 | 91.23 | 7.94 | 11.5 |
| | 15 | 19.9 | 5.73 | 5.10 | 1.1 |

The suitability of variant A2C8 and K7F11 as transamidases was further investigated in comparison to the native Trypsiligase II. Therefore the enzymatic parameters for the catalyzed transamidation reaction were determined by a model transamidation reaction using peptide substrates with the preferred recognition motive. In addition the model transamidation reaction was performed at high (250 μM) and low (15 μM) substrate concentrations to check, whether the new biocatalysts could also efficiently catalyze the desired reaction at low substrate concentration, which is a crucial requirement for the modification of therapeutic proteins. At high substrate concentrations the native Trypsiligase II shows only poor aminolysis to hydrolysis ratio of 0.9 which leads to an moderate product yield of 17%. A reduction of the substrate concentration to 15 μM leads to a significantly reduced apparent turnover rate for the aminolysis reaction, whereas the deacylation step is dominated by the hydrolysis reaction with a tenfold higher turnover rate, resulting in a poor product yield of 3%. The improved biocatalysts showed a dramatically increased synthesis efficiency, especially variant A2C8. At 250 μM substrate concentration A2C8 possess an excellent aminolysis to hydrolysis ratio of 70 ending up with an product yield of 64.9% that nearly fits with the theoretically reachable yield of 67% which is thermodynamically limited under the given reaction conditions (twofold excess of nucleophile). Even at a low substrate concentration the aminolysis reaction clearly dominates within the deacylation step showing a sevenfold higher apparent turnover rate than the hydrolysis reaction. Reaching a product yield of 46% A2C8 shows a 14 fold higher product yield than the native Trypsiligase II. It was assumed that an improved transamidation activity is reached by introducing mutations which improve the affinity of the biocatalyst for the nucleophilic peptide and/or reduce its hydrolysis activity. Therefore corresponding experimental data describing both parameters for the native Trypsiligase II as well as variant A2C8 were determined. A summary of these data is listed in table 7 and includes the enzyme's K_{M} values for the nucleophilic peptide as well as the turnover rate for the hydrolysis in the absence of a nucleophilic peptide.

A first indication that the assumption towards an improved affinity for the nucleophilic peptide already applies to the native trypsiligase II was observed in the study for the zinc dependence of the transamidation reaction catalyzed by Trypsiligase II. It was shown that Trypsiligase II has a strong dependence for zinc ions as the absence of zinc ions leads to a significant decrease of the apparent turnover rate for the aminolysis reaction and more important to a noticeable reduction of the product yield by a factor of 4. As mentioned above, the reason of the lowered enzyme affinity for the nucleophilic peptide is a missing complexation between the artificial histidins of Trypsiligase II (H39 and H59) and the peptide localized histidine in P₃′-position mediated by a zinc ion.

This zinc dependent influence on the affinity for Trypsiligase II was confirmed by measurements of the K_{M} values for the nucleophilic peptide in the presence or absence of zinc ions. In the presence of zinc ions the K_{M} value for the nucleophilic peptide is 143 μM. In the absence of zinc ions the K_{M} value for the nucleophilic peptide is increased by a factor of 11 to 1630 μM. Using identical substrate and reaction conditions, the variant A2C8 reaches a product yield of 59.7% independently from the presence of zinc ions. A key element for this enhanced transamidation reaction is based on a further improved enzyme affinity for the nucleophilic peptide. In the absence of zinc ions A2C8 has a K_{M} value of 17.6 μM for the nucleophilic peptide. In comparison to the native Trypsiligase II this corresponds to an improvement of the enzyme affinity for the nucleophilic peptide by a factor of 8 and 92 in the presence or absence of zinc ions, respectively.

A further key element for the enhanced transamidation reaction of A2C8 relies on its reduced intrinsic hydrolysis activity in comparison to the native Trypsiligase II. In the absence of a nucleophilic peptide variant A2C8 has a turnover rate for the hydrolysis reaction of 86.6 mkat/mol whereas the turnover rate of Trypsiligase II was determined with 602.1 mkat/mol. This reduction in the enzyme's hydrolysis activity by a factor of 7 is an important parameter as the hydrolysis competes with the aminolysis within the deacylation step determining the product yield of the synthesis reaction. To sum it up we showed that the enhanced transamidation behavior of A2C8 is a cause of an improved enzyme affinity for the nucleophilic peptide as well as a reduced intrinsic hydrolysis activity.

Those two features of A2C8 are directly related to the new mutations that were introduced into Trypsiligase II within our evolutionary approach.

TABLE 7
Summary of enzymatic parameters for Trypsiligase II as well as the
improved variant A2C8. The measurements for the turnover rate of
the hydrolysis reaction (k_{cat, HL}) as well as for the K_{M} values of
the nucleophilic peptide (acyl acceptor) were carried out with Bz-
PGGYRAHAG-OH used as acyl donor. K_{M} values for the acyl acceptor
were determined by measuring the apparent turnover rates for the
corresponding aminolysis reaction at a constant acyl donor
concentration and varying acyl acceptor concentrations. For
Trypsiligase II the measurements for K_{M} values of the nucleophilic
peptide were carried out both in the presence or absence of zinc
ions due to its strong zinc ion dependency. Hydrolysis reactions
were carried out at following conditions: varying concentrations
of Bz-PGGYRAHAG-OH (acyl donor), 2 μM trypsin variant, 100 mM
HEPES/NaOH pH 7.8, ±0.1 mM ZnCl₂, 100 mM NaCl, 10 mM CaCl₂,
T = 30° C. UPLC analysis conditions: Waters Acquity Ultra
Performance LC, C18-column, gradient 5-40% acetonitrile, 5 min,
detection at 254 nm; Transamidation reactions for K_{M} values were
carried out at following conditions: 250 μM Bz-PGGYRAHAG-OH
(acyl donor), varying concentrations of H-RAHAK(DNP)-OH (acyl
acceptor) 0.2-1.5 μM trypsin variant, 100 mM HEPES/NaOH pH
7.8, ±0.1 mM ZnCl₂, 100 mM NaCl, 10 mM CaCl₂, T = 30°
C. The maximum product yield was determined with two equivalents
of acyl acceptor and 250 μM acyl donor compound. UPLC analysis
conditions: Waters Acquity Ultra Performance LC, C18-column,
gradient 5-60% acetonitrile, 5 min, detection at 360 nm.

| | Zinc ions | product yield | k_{cat, HL} | K_{M, acyl acceptor} |
|---|---|---|---|---|
| Variant | (100 μM) | (%) | (mkat/mol) | (μM) |
| Trypsiligase II | no | 4.2 | n.d. | 1630 |
| Trypsiligase II | yes | 18.1 | 602.1 | 143.7 |
| A2C8 | no | 59.7 | 86.6 | 17.6 |

### Example 5

In order to demonstrate the activating effect of the mutations of A2C8 and K7F11 introduced by the evolutionary selection, in the next step mutations Y39H, Y59H, K60E and D189K derived from Trypsiligase II were gradually mutated back to the amino acid residues present in wild-type trypsin and the synthetic potential was analyzed. These data showed that the positions to be protected by this patent are sufficient to induce transamidation activity in wild-type trypsin, even without the mutations resulting from Trypsiligase II (Y39H/Y59H/K60E/D189K) or Trypsiligase I (K60E/N143H/E151H/D189K).

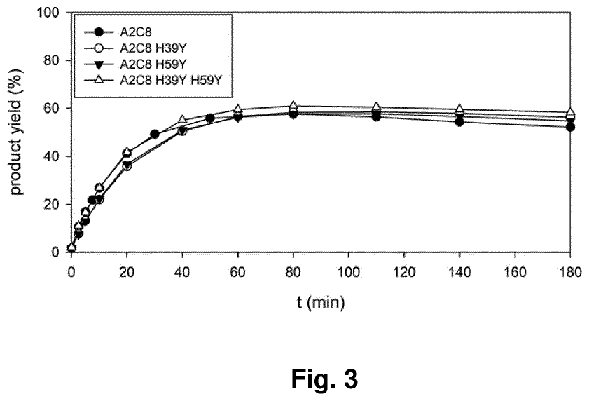

As the first step, influence for back mutations of the artificial histidines at Position 39 and 59 in A2C8 to tyrosine (which could be found for wild-type trypsin at these positions) was investigated by a model transamidation reaction. Variants with a single back mutation at position 39 or 59 (A2C8_H39Y and A2C8_H59Y) as well as the variant with mutations at both positions 39 and 59 (A2C8_H39Y/H59Y) have been investigated (FIG. 3).

Variants with a single back mutation at position 39 or 59 (A2C8_H39Y and A2C8_H59Y) as well as the variant with mutations at position 39 and 59 (A2C8_H39Y/H59Y) showed a comparable synthesis behavior as variant A2C8 (shown in FIG. 3). This leads to the conclusion that wild-type mutations at Position 39 and 59 have no influence on the synthesis efficiency of A2C8 indicating that the favorable synthesis properties of A2C8 were gained by the newly introduced mutations at the randomized positions.

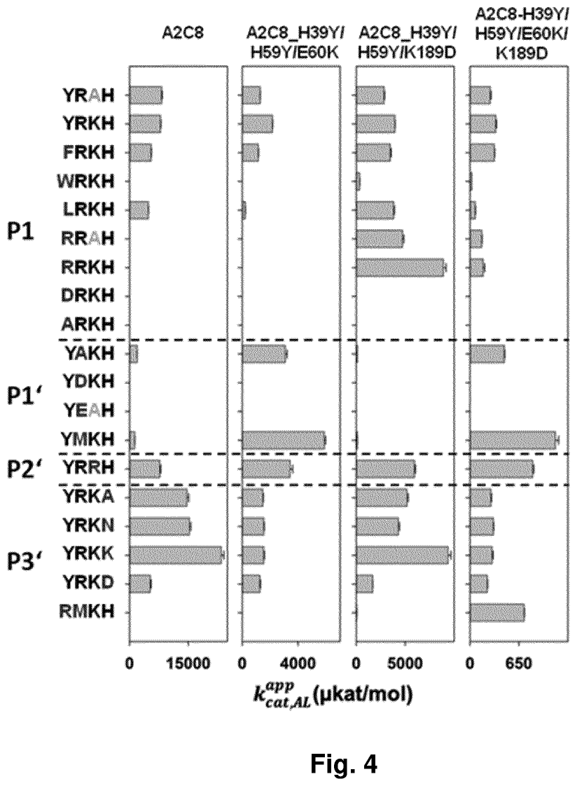

To further investigate if the identified mutations are sufficient to turn the wild-type protease trypsin into a transamidase, additionally the two further Trypsiligase II associated mutations at position 189 and 60 were mutated back to the wild-type amino acids. Back mutations at position 189 and 60 were introduced as single back mutation (A2C8_H39Y/H59Y/E60K and A2C8_H39Y/H59Y/K189D) as well as double mutation (A2C8_H39Y/H59Y/E60K/K189D) into variant A2C8_H39Y/H59Y. In a first step the substrate specificity was investigated for all variants as position 189 is known to influence the S₁ specificity of trypsin and position 60 is known to influence the S₁′ specificity (FIG. 4).

The back mutation at position 60 within variant A2C8_H39Y/H59Y/E60K effects more flexibility at P₁′-position of the substrate enabling also the acceptance of methionine or alanine. The back mutation at position 189 within variant A2C8_H39Y/H59Y/K189D effects more flexibility at P₁-position of the substrate enabling also the acceptance of arginine, which correlates with the specificity of wild-type trypsin. Variant A2C8_H39Y/H59Y/E60K/K189D which carries all four back mutations shows an specificity profile which combines the flexibility of A2C8_H39Y/H59Y/E60K in P₁′-position and A2C8_H39Y/H59Y/K189E at P₁-position of the substrate.

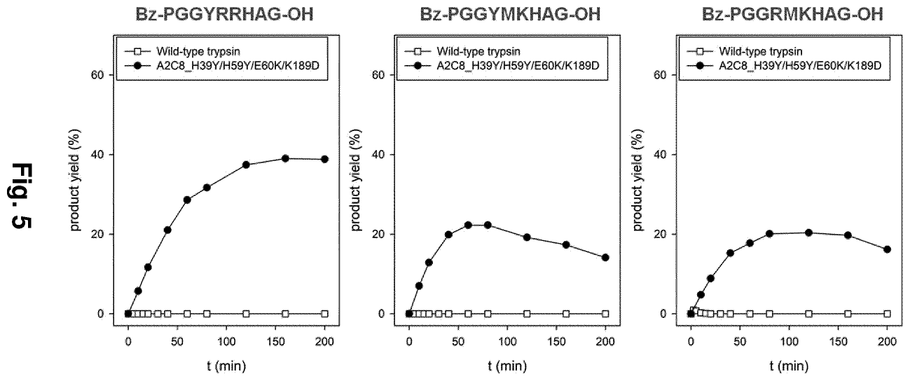

The three substrates with the highest apparent turnover rate for the aminolysis reaction were then used for investigating the synthesis behavior of variant A2C8_H39Y/H59Y/E60K/K189D. In FIG. 5 the time course of product formation for a transamidation reaction catalyzed by variant A2C8_H39Y/H59Y/E60K/K189D with various peptide substrates is illustrated.

As shown in FIG. 5 Variant A2C8_H39Y/H59Y/E60K/K189D is able to efficiently catalyze the formation of a transamidation product with substrates bearing the recognition sequence YRRH, YMKH and RMKH. The highest product yield could be observed for the substrate with the recognition sequence YRRH which was 38%. A2C8 reaches at comparable conditions a product yield of 59%. Nearly no product formation could be observed for wild-type trypsin.

This confirms the previous assumption, that the newly introduced mutations are sufficient enough to turn the wild-type protease trypsin into a transamidase.

As those mutations are associated with an improved enzyme affinity for the nucleophilic peptide as well as a reduced intrinsic hydrolysis activity it could be concluded, that all trypsin species could be converted into a transamidase in general by the introduction of amino acid exchanges that affect an improvement of the enzyme affinity for the nucleophilic peptide and/or reduce the intrinsic hydrolysis activity.

### Example 6

Subsequently a dual modification of a Fab-fragment was investigated by utilizing variation in substrate specificity of two Trypsiligase II variants.

Interestingly the back mutation of Trypsiligase II associated mutations at position 39, 59, and 189 within variant K7F11 (resulting variant K7F11_H39Y/H59Y/K189D) leads to a transamidase with alternated specificity in Pr-Position.

K7F11_H39Y/H59Y/K189D has a high specific activity for the recognition sequence RRKH whereas aromatic or aliphatic substitutions, which are at least accepted by A2C8 and K7F11, leads to significantly reduced apparent turnover rates for the aminolysis reaction.

This means, that variant K7F11_H39Y/H59Y/K189D could efficiently discriminate for the recognition sequence RRKH even in the presence of an A2C8 related recognition sequence like YRAH.

To prove the possibility for an orthogonal dual-modification of a protein upon two distinct modification sites a Her2 specific Fab-fragment was equipped with the RRKH-motif at the C-terminal end of the light chain and the YRAH-motif a at the C-terminal end of the heavy chain (see FIG. 6).

Following a sequential dual-modification was performed:

In a first step the light chain was modified with a Carboxyfluorescein bearing nucleophile which was catalyzed by variant K7F11_H39Y/H59Y/K189D. Analysis by mass spectrometry revealed a nearly exclusively modification at the C-terminal end of the light chain. The enzyme as well as the remaining nucleophile (RKHAK(CF)-OH) were removed by Protein G affinity chromatography.

In a second step the heavy chain was modified with a DM1-bearing nucleophile which was catalyzed by variant A2C8. An exclusively modification at the C-terminal end of the heavy chain could be confirmed by mass spectrometry. The yield of dual labeled Fab-fragment was approximately 75% as estimated by mass spectrometry.

In the context of an orthogonal dual-modification, whereas the term “orthogonal” refers to the modification of a polypeptide upon two varying recognition sequences with two different biocatalysts of the same origin without significant cross reactivity, following modification strategies including the N- and C-terminal localization of recognition sequences could be possible (see Table 8).

TABLE 8
Summary of possible enzyme and recognition sequence pairs within
the scope of dual and orthogonal modification of proteins.

| Nr. | Enzyme 1 | Sequence 1 | Enzyme 2 | Sequence 2 |
|---|---|---|---|---|
| 1 | A2C8 | LRKH | K7F11 | WRAH |
| | | (C-terminal) | | (C-terminal) |
| 2 | A2C8 | YRAH | K7F11_H39Y/ | RRKH |
| | | (C-terminal) | H59Y/K189D | (C-terminal) |
| 3 | Trypsiligase II | RAH | A2C8 | YRKK |
| | | (N-terminal) | | (C-terminal) |
| 4 | Trypsiligase II | RAH | K7F11 | YRKK |
| | | (N-terminal) | | (C-terminal) |
| 5 | K7F11_H39Y/ | RKH | A2C8 | YRAH |
| | H59Y/K189D | (N-terminal) | | (C-terminal) |
| 6 | K7F11_H39Y/ | RKH | K7F11 | YRAH |
| | H59Y/K189D | (N-terminal) | | (C-terminal) |

The most important key data for the relevant Trypsiligase variants or libraries are summarized in the following table 9:

TABLE 9
Summary of data for Trypsiligase variants or libraries described herein.

| | | | |
|---|---|---|---|
| | Mutation in comparison to Rat | Randomized | |
| Designation | anionic trypsin II wild-type | positions | Remarks |

Trypsiligase I

| | | | |
|---|---|---|---|
| Trypsiligase I | K60E, N143H, E151H, D189K | — | Recognition |
| | | | sequence YRH |
| Trypsiligase | K60E, N143H, E151H, D189K | D95, R96, L99, | |
| I-library B | | S214, G219, | |
| | | A221 | |
| 2G10 | K60E, N143H, E151H, D189K, H40P, | — | From Trypsiligase |
| | A55S, K97D, L99F, S190S, Q192E | | I-library A, high |
| | | | nucleophile-affinity, |
| | | | YRH |
| 1C11 | K60E, N143H, E151H, D189K, D95D, | — | From Trypsiligase |
| | R96V, L99F, S214G, G219S, A221G | | I-library B, low |
| | | | hydrolysis activity, |
| | | | YRH |
| Hybrid | K60E, N143H, E151H, D189K, H40P, | | Hybrid variant |
| | A55S, D95D, R96V, K97D, L99F, | | combining |
| | S190S, Q192E, S214G, G219S, | | mutations of 2G10 |
| | A221G | | and 1C11 |

Trypsiligase II

| | | | |
|---|---|---|---|
| Trypsiligase II | Y39H, Y59H, K60E, D189K | — | Recognition |
| | | | sequence YRAH |
| Trypsiligase | Y39H, Y59H, K60E, D189K, L99F | H40, A55, R96, | |
| II-library | | K97, N143, | |
| | | E151, S190, | |
| | | Q192, S214, | |
| | | G219, A221 | |
| A2C8 | Y39H, Y59H, K60E, D189K, H40F, | — | From Trypsiligase |
| | A55A, R96E, K97D, L99F, N143E, | | II-library |
| | E151Y, S190V, Q192A, S214G, | | |
| | G219Q, A221T | | |
| A2C8_H39Y | Y59H, K60E, D189K, H40F, A55A, | — | |
| | R96E, K97D, L99F, N143E, E151Y, | | |
| | S190V, Q192A, S214G, G219Q, | | |
| | A221T | | |
| A2C8_H59Y | Y39H, K60E, D189K, H40F, A55A, | — | |
| | R96E, K97D, L99F, N143E, E151Y, | | |
| | S190V, Q192A, S214G, G219Q, | | |
| | A221T | | |
| A2C8_H39Y/H59Y | K60E, D189K, H40F, A55A, R96E, | — | |
| | K97D, L99F, N143E, E151Y, S190V, | | |
| | Q192A, S214G, G219Q, A221T | | |
| A2C8_H39Y/H59Y/ | D189K, H40F, A55A, R96E, K97D, | — | |
| E60K | L99F, N143E, E151Y, S190V, Q192A, | | |
| | S214G, G219Q, A221T | | |
| A2C8_H39Y/H59Y/ | K60E, H40F, A55A, R96E, K97D, | — | |
| K189D | L99F, N143E, E151Y, S190V, Q192A, | | |
| | S214G, G219Q, A221T | | |
| A2C8_H39Y/H59Y/ | H40F, A55A, R96E, K97D, L99F, | — | |
| E60K/K189D | N143E, E151Y, S190V, Q192A, | | |
| | S214G, G219Q, A221T | | |
| K7F11 | Y39H, Y59H, K60E, D189K, H40Y, | — | From Trypsiligase |
| | A55A, R96E, K97E, L99F, N143V, | | II-library |
| | E151E, S190A, Q192V, S214G, | | |
| | G219P, A221Q | | |
| K7F11_H39Y | Y59H, K60E, D189K, H40Y, A55A, | — | |
| | R96E, K97E, L99F, N143V, E151E, | | |
| | S190A, Q192V, S214G, G219P, | | |
| | A221Q | | |
| K7F11_H59Y | Y39H, K60E, D189K, H40Y, A55A, | — | |
| | R96E, K97E, L99F, N143V, E151E, | | |
| | S190A, Q192V, S214G, G219P, | | |
| | A221Q | | |
| K7F11_H39Y/H59Y | K60E, D189K, H40Y, A55A, R96E, | — | |
| | K97E, L99F, N143V, E151E, S190A, | | |
| | Q192V, S214G, G219P, A221Q | | |
| K7F11_H39Y/H59Y/ | D189K, H40Y, A55A, R96E, K97E, | — | |
| E60K | L99F, N143V, E151E, S190A, Q192V, | | |
| | S214G, G219P, A221Q | | |
| K7F11_H39Y/H59Y/ | K60E, H40Y, A55A, R96E, K97E, | — | |
| K189D | L99F, N143V, E151E, S190A, Q192V, | | |
| | S214G, G219P, A221Q | | |
| K7F11_H39Y/H59Y/ | H40Y, A55A, R96E, K97E, L99F, | — | |
| E60K/K189D | N143V, E151E, S190A, Q192V, | | |
| | S214G, G219P, A221Q | | |

The following sequences are disclosed herein:

Rat anionic trypsin H (SEQ ID NO: 1):
IVGGYTCQEN SVPYQVSLNS GYHFCGGSLI NDQWVVSAAH
CYKSRIQVRL GEHNINVLEG NEQFVNAAKI IKHPNFDRKT
LNNDIMLIKL SSPVKLNARV ATVALPSSCA PAGTQCLISG
WGNTLSSGVN EPDLLQCLDA PLLPQADCEA SYPGKITDNM
VCVGFLEGGK DSCQGDSGGP VVCNGELQGI VSWGYGCALP
DNPGVYTKVC NYVDWIQDTI AAN
anti-Her2-Fab-LC_RRKH/HC_YRAH, Heavy chain
(SEQ ID NO: 2):
EVKLQESGGG LVQPGGSLRL SCAASGFNIK DTYIHWVRQA
PGKGLEWVAR IYPTNGYTRY ADSVKGRFTI SADTSKNTAY
LQMNSLRAED TAVYYCSRWG GDGFYAMDYW GQGTLVTVSS
ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKKVEP KSCADKPGG YRAHAGEQKL
ISEEDL
anti-Her2-Fab-LC_RRKH/HC_YRAH, Light chain
(SEQ ID NO: 3):
DIELTQSPSS LSASVGDRVT ITCRASQDVN TAVAWYQQKP
GKAPKLLIYS ASFLYSGVPS RFSGSRSGTD FTLTISSLQP
EDFATYYCQQ HYTTPPTFGQ GTKLEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGECLSPGGR RKHAG

Further the following polypeptides are disclosed:

| SEQ ID NO | Peptide sequence |
|---|---|
| 4 | Bz-PGGYRAHAG-OH |
| 5 | Bz-PGGFRAHAG-OH |
| 6 | Bz-PGGWRAHAG-OH |
| 7 | Bz-PGGLRAHAG-OH |
| 8 | Bz-PGGDRAHAG-OH |
| 9 | Bz-PGGRRAHAG-OH |
| 10 | Bz-PGGARAHAG-OH |
| 11 | Bz-PGGYAAHAG-OH |
| 12 | Bz-PGGYDAHAG-OH |
| 13 | Bz-PGGYEAHAG-OH |
| 14 | Bz-PGGYKAHAG-OH |
| 15 | Bz-PGGYRAAAG-OH |
| 16 | Bz-PGGYRANAG-OH |
| 17 | Bz-PGGYRAKAG-OH |
| 18 | Bz-PGGYRADAG-OH |
| 19 | Bz-PGGYRKHAG-OH |
| 20 | Bz-PGGFRKHAG-OH |
| 21 | Bz-PGGWRKHAG-OH |
| 22 | Bz-PGGLRKHAG-OH |
| 23 | Bz-PGGDRKHAG-OH |
| 24 | Bz-PGGRRKHAG-OH |
| 25 | Bz-PGGARKHAG-OH |
| 26 | Bz-PGGYAKHAG-OH |
| 27 | Bz-PGGYDKHAG-OH |
| 28 | Bz-PGGYMKHAG-OH |
| 29 | Bz-PGGYRRHAG-OH |
| 30 | Bz-PGGYRKAAG-OH |
| 31 | Bz-PGGYRKNAG-OH |
| 32 | Bz-PGGYRKKAG-OH |
| 33 | Bz-PGGYRKDAG-OH |
| 34 | Bz-PGGYKRKAG-OH |
| 35 | Bz-PGGRMKHAG-OH |
| 36 | H-RAHAK(DNP)-OH |
| 37 | H-AAHAK(DNP)-OH |
| 38 | H-DAHAK(DNP)-OH |
| 39 | H-EAHAK(DNP)-OH |
| 40 | H-KAHAK(DNP)-OH |
| 41 | H-RAAAK(DNP)-OH |
| 42 | H-RANAK(DNP)-OH |
| 43 | H-RAKAK(DNP)-OH |
| 44 | H-RADAK(DNP)-OH |
| 45 | H-RKHAK(DNP)-OH |
| 46 | H-AKHAK(DNP)-OH |
| 47 | H-DKHAK(DNP)-OH |
| 48 | H-MKHAK(DNP)-OH |
| 49 | H-RRHAK(DNP)-OH |
| 50 | H-RKAAK(DNP)-OH |
| 51 | H-RKNAK(DNP)-OH |
| 52 | H-RKKAK(DNP)-OH |
| 53 | H-RKDAK(DNP)-OH |
| 54 | H-KRKAK(DNP)-OH |
| 55 | H-RAHAK-OH |
| 56 | H-RKHAK-OH |
| 57 | H-RKKAK-OH |
| 58 | H-RKAAK-OH |
| 59 | H-KRKAK-OH |
| 60 | H-RKHAK(CF)-OH |
| 61 | H-RKKAK(HexMal-PEG20.000)AAK(DNP)-OH |
| 62 | H-RKKAK(MCC-DM1)-OH |
| 63 | H-RKKAK(Ac-DM1)-OH |

Gene sequence of anti-Her2-Fab-LC_RRKH/HC_YRAH (SEQ ID NO: 64):
ATGAAGAAAACCGCGATTGCGATTGCGGTGGCGCTGGCGGGCTTTGCGACCGTGGCGCAG
GCGGATATTGAACTGACCCAGAGCCCGAGCAGCCTGAGCGCGAGCGTGGGCGATCGCGTG
ACCATTACCTGCCGCGCGAGCCAGGATGTGAACACCGCGGTGGCGTGGTATCAGCAGAAA
CCGGGCAAAGCGCCGAAACTGCTGATTTATAGCGCGAGCTTTCTGTATAGCGGCGTGCCG
AGCCGCTTTAGCGGCAGCCGCAGCGGCACCGATTTTACCCTGACCATTAGCAGCCTGCAG
CCGGAAGATTTTGCGACCTATTATTGCCAGCAGCATTATACCACCCCGCCGACCTTTGGC
CAGGGCACCAAACTGGAAATTAAACGCACCGTGGCGGCGCCGAGCGTGTTTATTTTTCCG
CCGAGCGATGAACAGCTGAAAAGCGGCACCGCGAGCGTGGTGTGCCTGCTGAACAACTTT
TATCCGCGCGAAGCGAAAGTGCAGTGGAAAGTGGATAACGCGCTGCAGAGCGGCAACAGC
CAGGAAAGCGTGACCGAACAGGATAGCAAAGATAGCACCTATAGCCTGAGCAGCACCCTG
ACCCTGAGCAAAGCGGATTATGAAAAACATAAAGTGTATGCGTGCGAAGTGACCCATCAG
GGCCTGAGCAGCCCGGTGACCAAATCTTTTAACCGCGGCGAATGCCTGAGCCCCGGAGGA
CGCCGCAAACATGCGGGCTGAGGAGGAAAAAAAAATGAAAAAGACAGCTATCGCAATTGC
AGTGGCGCTAGCTGGTTTCGCCACCGTGGCGCAAGCTGAAGTGAAACTGCAGGAAAGCGG
TGGTGGTCTGGTGCAGCCGGGTGGTAGCCTGCGCCTGAGCTGCGCGGCGAGCGGCTTTAA
CATTAAAGATACCTATATTCATTGGGTGCGCCAGGCGCCGGGCAAAGGCCTGGAATGGGT
GGCGCGCATTTATCCGACCAACGGCTATACCCGCTATGCGGATAGCGTGAAAGGCCGCTT
TACCATTAGCGCGGATACCAGCAAAAACACCGCGTATCTGCAGATGAACAGCCTGCGCGC
GGAAGATACCGCGGTGTATTATTGCAGCCGCTGGGGCGGCGATGGCTTTTATGCGATGGA
TTATTGGGGCCAGGGCACCCTGGTGACCGTGAGCAGCGCGAGCACCAAAGGCCCGAGCGT
GTTTCCGCTGGCGCCGAGCAGCAAAAGCACCAGCGGCGGCACCGCGGCGCTGGGCTGCCT
GGTGAAAGATTATTTTCCGGAACCGGTGACCGTGAGCTGGAACAGCGGCGCGCTGACCAG
CGGCGTGCATACCTTTCCGGCGGTGCTGCAGAGCAGCGGCCTGTATAGCCTGAGCAGCGT
GGTGACCGTGCCGAGCAGCAGCCTGGGCACCCAGACCTATATTTGCAACGTGAACCATAA
ACCGAGCAACACCAAAGTGGATAAAAAAAGTGGAACCGAAAAGCTGCGCGGATAAACCCGG
AGGATATCGCGCGCATGCGGGCGAACAGAAACTGATTAGCGAAGAAGATCTG
DNP—2,4-dinitrophenyl
CF—5(6)-Carboxyfluoresceine
HexMal—6-Maleimidohexanoic add
PEG20.000—Polyethylene glycol with average mass of 20000 g/mol
MCC—4-(N-Maleimidomethyl)cyclohexane-1-carboxylate
Ac—Acetate linker

1. A mutated trypsin comprising an amino acid substitution at least at two amino acid positions leading to an increased affinity for the nucleophilic substrate and/or at least at two amino acid positions leading to a reduced hydrolysis activity.

2. A mutated trypsin according to claim 1, comprising
an amino acid substitution at least at two amino acid positions selected from group 1 comprising H40, A55, S214, G219, A221, preferably further comprising an amino acid substitution at least at one amino acid position of group 2 comprising R96, K97, L99, N143, E151, S190, Q192; or
an amino acid substitution at least at one amino acid position selected from group 1 comprising H40, A55, S214, G219, A221 and an amino acid substitution at least at one amino acid position of group 2 comprising R96, K97, L99, N143, E151, S190, Q192; or
an amino acid substitution at least at one amino acid position selected from group 1 comprising H40, A55, S214, G219, A221 and an amino acid substitution at least at two amino acid position of group 2 comprising R96, K97, L99, N143, E151, S190, Q192; or
an amino acid substitution at least at two amino acid position selected from group 1 comprising H40, A55, S214, G219, A221 and an amino acid substitution at least at two amino acid position of group 2 comprising R96, K97, L99, N143, E151, S190, Q192; or
an amino acid substitution at least at three amino acid position selected from group 1 comprising H40, A55, S214, G219, A221 and an amino acid substitution at least at one amino acid position of group 2 comprising R96, K97, L99, N143, E151, S190, Q192; or
an amino acid substitution at least at three amino acid position selected from group 1 comprising H40, A55, S214, G219, A221 and an amino acid substitution at least at two amino acid position of group 2 comprising R96, K97, L99, N143, E151, S190, Q192; or
an amino acid substitution at least at three amino acid position selected from group 1 comprising H40, A55, S214, G219, A221 and an amino acid substitution at least at three amino acid position of group 2 comprising R96, K97, L99, N143, E151, S190, Q192.

3. The mutated trypsin according to claim 1, wherein;
the amino acid at position 40 is aromatic, preferably F or Y; and/or
the amino acid at position 55 is a small aliphatic polar amino acid, preferably A, V, S, or T; and/or
the amino acid at position 96 is an acidic, polar amino acid, preferably E or P; and/or
the amino acid at position 97 is preferably H, D, or F; and/or
the amino acid at position 99 is an aromatic amino acid, preferably F, Y, W or M; and/or
the amino acid at position 143 is preferably V, D, E or T; and/or
the amino acid at position 151 is preferably D, A, T, or Y; and/or
the amino acid at position 190 is a small aliphatic amino acid, preferably A or V; and/or
the amino acid at position 192 is a small aliphatic or aromatic amino acid, preferably A, V, F, or W; and/or
the amino acid at position 214 is a small aliphatic amino acid, preferably G or A; and/or
the amino acid at position 219 is a polar amino acid, preferably Q or P; and/or
the amino acid at position 221 is a polar amino acid, preferably Q or T.

4-14. (canceled)

15. The mutated trypsin according to claim 1, further comprising additional amino acid substitutions at both position K60 and D189, and at least one more amino acid substitution at position Y39 or Y59.

16. The mutated trypsin of claim 15, wherein position Y39 and position Y59 are substituted.

17. The mutated trypsin of claim 15, wherein position K60 is substituted by E or D.

18. The mutated trypsin of claim 15, wherein position D189 is substituted by K, H or R.

19. The mutated trypsin of claim 15, wherein position Y39 is substituted by K, H or R.

20. The mutated trypsin of claim 15, wherein position Y59 is substituted by K, H or R.

21. The mutated trypsin of claim 1, further comprising additional amino acid substitutions K60E, D189K, N143H, and E151H.

22. The mutated trypsin according to claim 1, further comprising additional amino acid substitutions both at position K60 and D 189, and at least one more amino acid substitution by histidine at position N143 or position E151.

23. The mutated trypsin of claim 22, wherein K60 is substituted by E or D.

24. The mutated trypsin of claim 22, wherein D 189 is substituted by K, H or R.

25. The mutated trypsin of claim 1, further comprising further comprising additional amino acid substitutions Y39H, Y59H, K60E, and D189K.

26. (canceled)

27. A method for orthogonal dual-modification of a substrate comprising the following steps:
a) providing a substrate for orthogonal dual-modification
b) modifying the substrate using a first trypsin enzyme recognizing a first recognition sequence,
c) modifying the substrate using a second trypsin enzyme recognizing a second recognition sequence.

28. The method according to claim 27, wherein the first or second trypsin enzyme is selected from the group comprising Trypsiligase II, trypsin variant A2C8, trypsin variant K7F11, trypsin variant K7F11_H39Y/H59Y/K189D.
